(19) United States
(12) Patent Application Publication
(10) Pub. No.: US 2024/0316544 A1
(43) Pub. Date: Sep. 26, 2024

(54) Processes for Regenerating Catalysts and for Upgrading Alkanes and/or Alkyl Aromatic Hydrocarbons

(71) Applicant: ExxonMobil Chemical Patents Inc., Baytown, TX (US)

(72) Inventor: Xiaoying Bao, Houston, TX (US)

(21) Appl. No.: 18/579,568

(22) PCT Filed: Jul. 25, 2022
(86) PCT No.: PCT/US2022/038131

§ 371 Date: Jan. 16, 2024

Related U.S. Application Data
(60) Provisional application No. 63/231,946, filed on Aug. 11, 2021.

(51) Int. Cl. B01J 38/12; B01J 21/06; B01J 21/08; B01J 23/42; B01J 23/90; C07C 5/32

(52) U.S. Cl. CPC B01J 38/12; B01J 21/066; B01J 21/08; B01J 23/42; B01J 23/90; C07C 5/325; C07C 2521/06; C07C 2521/08; C07C 2523/42

(57) ABSTRACT

Processes for regenerating an at least partially deactivated catalyst that can include a Group (10) element, an inorganic support, and a contaminant. The Group (10) element can have a concentration of from 0.06 wt % to 6 wt %, based on the weight of the inorganic support. The process can include (I) heating the deactivated catalyst using a heating gas mixture that includes H₂O at a concentration >5 mol %, based on the total moles in the mixture to produce a precursor catalyst. The process can also include (II) providing an oxidative gas that includes ≤5 mol % of H₂O, based on the total moles in the oxidative gas, and (III) contacting the precursor catalyst at an oxidizing temperature with the oxidative gas for a duration of at least 30 seconds to produce an oxidized precursor catalyst. The process can also include (IV) obtaining a regenerated catalyst from the oxidized precursor catalyst.

### CROSS-REFERENCE TO RELATED APPLICATION

This application claims priority to and the benefit of U.S. Provisional Application No. 63/231,946 having a filing date of Aug. 11, 2021, the disclosure of which is incorporated herein by reference in its entirety.

### FIELD

This disclosure relates to processes for regenerating a catalyst and for upgrading alkanes and/or alkyl aromatic hydrocarbons.

### BACKGROUND

Catalytic dehydrogenation, dehydroaromatization, and dehydrocyclization of alkane and/or alkyl aromatic hydrocarbons are industrially important chemical conversion processes that are endothermic and equilibrium-limited. The dehydrogenation of alkanes, e.g., C₂-C₁₂ alkanes, and/or alkyl aromatics, e.g., ethylbenzene, can be done through a variety of different supported catalyst systems such as the Pt-based, Cr-based, Ga-based, V-based, Zr-based, In-based, W-based, Mo-based, Zn-based, and Fe-based systems. Among the existing propane dehydrogenation processes, certain process uses an alumina supported chromia catalyst that provides one of the highest propylene yields of approximately 50% (55% propane conversion at 90% propylene selectivity), which is obtained at a temperature of approximately 560° C. to 650° C. and at a low pressure of 20 kPa-absolute to 50 kPa-absolute. It is desirable to increase the propylene yield without having to operate at such low pressure to increase the efficiency of the dehydrogenation process.

Increasing the temperature of the dehydrogenation process is one way to increase the conversion of the process according to the thermodynamics of the process. For example, at 670° C., 100 kPa-absolute, in the absence of any inert/diluent, the equilibrium yield propylene yield has been estimated via simulation to be approximately 74%. At such high temperature, however, the catalyst deactivates very rapidly and/or the propylene selectivity becomes uneconomically low. The rapid catalyst deactivation is believed to be caused by coke depositing onto the catalyst and/or agglomeration of the active phase. Coke can be removed by combustion using an oxygen-containing gas, however, agglomeration of the active phase is believed to be exacerbated during the combustion process, which rapidly reduces the activity and stability of the catalyst.

There is a need, therefore, for improved processes for regenerating at least partially deactivated catalysts and processes for dehydrogenating, dehydroaromatizing, and/or dehydrocyclizing alkane and/or alkyl aromatic hydrocarbons. This disclosure satisfies this and other needs.

### SUMMARY

Processes for regenerating an at least partially deactivated catalyst and processes for upgrading a hydrocarbon are provided. In some embodiments, the process can be used to regenerate an at least partially deactivated catalyst that can include a Group 10 element, an inorganic support, and a contaminant. The Group 10 element can have a concentration in the range from 0.06 wt % to 6 wt %, based on the weight of the inorganic support. The process can include (I) heating the at least partially deactivated catalyst using a heating gas mixture that can include H₂O at a concentration of greater than 5 mol %, based on the total moles in the heating gas mixture to produce a precursor catalyst. The process can also include (II) providing an oxidative gas that can include no greater than 5 mol % of H₂O, based on the total moles in the oxidative gas. The process can also include (III) contacting the precursor catalyst at an oxidizing temperature in a range from 620° C. to 1,000° C. with the oxidative gas for a duration of at least 30 seconds, preferably at least 1 minute, preferably at least 5 minutes, to produce an oxidized precursor catalyst. The process can also include (IV) obtaining a regenerated catalyst from the oxidized precursor catalyst.

In other embodiments, a process for upgrading a hydrocarbon can include (I) contacting a hydrocarbon-containing feed with a catalyst that can include a Group 10 element and an inorganic support to effect one or more of dehydrogenation, dehydroaromatization, and dehydrocyclization of at least a portion of the hydrocarbon-containing feed to produce an at least partially deactivated catalyst that can include the Group 10 element, the inorganic support, and a contaminant and an effluent that can include one or more upgraded hydrocarbons and molecular hydrogen. The hydrocarbon-containing feed can include one or more of C₂-C₁₆ linear or branched alkanes, or one or more of C₄-C₁₆ cyclic alkanes, one or more C₈-C₁₆ alkyl aromatics, or a mixture thereof. The Group 10 element can have a concentration in the range from 0.06 wt % to 6 wt %, based on the weight of the inorganic support. The hydrocarbon-containing feed and the catalyst can be contacted at a temperature in a range from 300° C. to 900° C. The one or more upgraded hydrocarbons can include at least one of a dehydrogenated hydrocarbon, a dehydroaromatized hydrocarbon, and a dehydrocyclized hydrocarbon. The process can also include (II) heating the at least partially deactivated catalyst using a heating gas mixture that can include H₂O at a concentration of greater than 5 mol %, based on the total moles in the heating gas mixture to produce a precursor catalyst. The process can also include (III) providing an oxidative gas that can include no greater than 5 mol % of H₂O, based on the total moles in the oxidative gas. The process can also include (IV) contacting the precursor catalyst at an oxidizing temperature in a range from 620° C. to 1,000° C. with the oxidative gas for a duration of at least 30 seconds to produce an oxidized precursor catalyst. The process can also include (V) obtaining a regenerated catalyst from the oxidized precursor catalyst. The process can also include (VI) contacting an additional quantity of the hydrocarbon-containing feed with at least a portion of the regenerated catalyst to produce additional at least partially deactivated catalyst and additional effluent.

### BRIEF DESCRIPTION OF THE DRAWINGS

FIG. 1 shows that the performance of a catalyst used in Example 5 for PDH was stable after 75+ cycles.

FIG. 2 shows that the performance of the comparative catalyst 1 continued to deactivate even though the regeneration temperature (620° C.) was much lower than the other examples.

### DETAILED DESCRIPTION

Various specific embodiments, versions and examples of the invention will now be described, including preferred embodiments and definitions that are adopted herein for purposes of understanding the claimed invention. While the following detailed description gives specific preferred embodiments, those skilled in the art will appreciate that these embodiments are exemplary only, and that the invention may be practiced in other ways. For purposes of determining infringement, the scope of the invention will refer to any one or more of the appended claims, including their equivalents, and elements or limitations that are equivalent to those that are recited. Any reference to the “invention” may refer to one or more, but not necessarily all, of the inventions defined by the claims.

In this disclosure, a process is described as comprising at least one “step.” It should be understood that each step is an action or operation that may be carried out once or multiple times in the process, in a continuous or discontinuous fashion. Unless specified to the contrary or the context clearly indicates otherwise, multiple steps in a process may be conducted sequentially in the order as they are listed, with or without overlapping with one or more other steps, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step. Preferably, the steps are conducted in the order described.

Unless otherwise indicated, all numbers indicating quantities in this disclosure are to be understood as being modified by the term “about” in all instances. It should also be understood that the precise numerical values used in the specification and claims constitute specific embodiments. Efforts have been made to ensure the accuracy of the data in the examples. However, it should be understood that any measured data inherently contains a certain level of error due to the limitation of the technique and/or equipment used for acquiring the measurement.

Certain embodiments and features are described herein using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated.

The indefinite article “a” or “an”, as used herein, means “at least one” unless specified to the contrary or the context clearly indicates otherwise. Thus, embodiments using “a reactor” or “a conversion zone” include embodiments where one, two or more reactors or conversion zones are used, unless specified to the contrary or the context clearly indicates that only one reactor or conversion zone is used.

The term “hydrocarbon” means (i) any compound consisting of hydrogen and carbon atoms or (ii) any mixture of two or more such compounds in (i). The term “Cn hydrocarbon,” where n is a positive integer, means (i) any hydrocarbon compound comprising carbon atom(s) in its molecule at the total number of n, or (ii) any mixture of two or more such hydrocarbon compounds in (i). Thus, a C2 hydrocarbon can be ethane, ethylene, acetylene, or mixtures of at least two of these compounds at any proportion. A “Cm to Cn hydrocarbon” or “Cm−Cn hydrocarbon,” where m and n are positive integers and m<n, means any of Cm, Cm+1, Cm+2, . . . , Cn−1, Cn hydrocarbons, or any mixtures of two or more thereof. Thus, a “C2 to C3 hydrocarbon” or “C2-C3 hydrocarbon” can be any of ethane, ethylene, acetylene, propane, propene, propyne, propadiene, cyclopropane, and any mixtures of two or more thereof at any proportion between and among the components. A “saturated C2-C3 hydrocarbon” can be ethane, propane, cyclopropane, or any mixture thereof of two or more thereof at any proportion. A “Cn+ hydrocarbon” means (i) any hydrocarbon compound comprising carbon atom(s) in its molecule at the total number of at least n, or (ii) any mixture of two or more such hydrocarbon compounds in (i). A “Cn− hydrocarbon” means (i) any hydrocarbon compound comprising carbon atoms in its molecule at the total number of at most n, or (ii) any mixture of two or more such hydrocarbon compounds in (i). A “Cm hydrocarbon stream” means a hydrocarbon stream consisting essentially of Cm hydrocarbon(s). A “Cm−Cn hydrocarbon stream” means a hydrocarbon stream consisting essentially of Cm−Cn hydrocarbon(s).

For the purposes of this disclosure, the nomenclature of elements is pursuant to the version of the Periodic Table of Elements (under the new notation) as provided in Hawley's Condensed Chemical Dictionary, 16^{th} Ed., John Wiley & Sons, Inc., (2016), Appendix V. For example, a Group 2 element includes Mg, a Group 4 element includes Zr, a Group 8 element includes Fe, a Group 9 element includes Co, a Group 10 element includes Ni, and a Group 13 element includes Al. The term “metalloid”, as used herein, refers to the following elements: B, Si, Ge, As, Sb, Te, and At. In this disclosure, when a given element is indicated as present, it can be present in the elemental state or as any chemical compound thereof, unless it is specified otherwise or clearly indicated otherwise by the context.

The term “alkane” means a saturated hydrocarbon. The term “cyclic alkane” means a saturated hydrocarbon comprising a cyclic carbon ring in the molecular structure thereof. An alkane can be linear, branched, or cyclic.

The term “aromatic” is to be understood in accordance with its art-recognized scope, which includes alkyl substituted and unsubstituted mono- and polynuclear compounds.

The term “rich” when used in phrases such as “X-rich” or “rich in X” means, with respect to an outgoing stream obtained from a device, e.g., a conversion zone, that the stream comprises material X at a concentration higher than in the feed material fed to the same device from which the stream is derived. The term “lean” when used in phrases such as “X-lean” or “lean in X” means, with respect to an outgoing stream obtained from a device, e.g., a conversion zone, that the stream comprises material X at a concentration lower than in the feed material fed to the same device from which the stream is derived.

The term “selectivity” refers to the production (on a carbon mole basis) of a specified compound in a catalytic reaction. As an example, the phrase “an alkane hydrocarbon conversion reaction has a 100% selectivity for an olefin hydrocarbon” means that 100% of the alkane hydrocarbon (carbon mole basis) that is converted in the reaction is converted to the olefin hydrocarbon. When used in connection with a specified reactant, the term “conversion” means the amount of the reactant consumed in the reaction. For example, when the specified reactant is propane, 100% conversion means 100% of the propane is consumed in the reaction. In another example, when the specified reactant is propane, if one mole of propane convers to one mole of methane and one mole of ethylene, the selectivity to methane is 33.3% and the selectivity to ethylene is 66.7%. Yield (carbon mole basis) is conversion times selectivity.

### Hydrocarbon Upgrading and Catalyst Regeneration Process

The hydrocarbon-containing feed can be or can include, but is not limited to, one or more alkane hydrocarbons, e.g., C₂-C₁₆ linear or branched alkanes and/or C₄-C₁₆ cyclic alkanes, and/or one or more alkyl aromatic hydrocarbons, e.g., C₈-C₁₆ alkyl aromatics. In some embodiments, the hydrocarbon-containing feed can optionally include 0.1 vol % to 50 vol % of steam, based on a total volume of any C₂-C₁₆ alkanes and any C₈-C₁₆ alkyl aromatics in the hydrocarbon-containing feed. In other embodiments, the hydrocarbon-containing feed can include <0.1 vol % of steam or can be free of steam, based on the total volume of any C₂-C₁₆ alkanes and any C₈-C₁₆ alkyl aromatics in the hydrocarbon-containing feed. The hydrocarbon-containing feed can be contacted with a catalyst that includes a Group 10 element, e.g., Pt, and an inorganic support, to effect one or more of dehydrogenation, dehydroaromatization, and dehydrocyclization of at least a portion of the hydrocarbon-containing feed to produce an at least partially deactivated catalyst that includes the Group 10 element, the inorganic support, and a contaminant, e.g., coke, and an effluent that can include one or more upgraded hydrocarbons and molecular hydrogen.

The one or more upgraded hydrocarbons can be or can include, but are not limited to, one or more dehydrogenated hydrocarbons, one or more dehydroaromatized hydrocarbons, one or more dehydrocylized hydrocarbons, or a mixture thereof. The hydrocarbon-containing feed and catalyst can be contacted at a temperature in a range from 300° C. to 900° C. In some embodiments, the hydrocarbon-containing feed and catalyst can be contacted for a time period of ≤5 hours, ≤4 hours, or ≤3 hours, ≤1 hour, ≤0.5 hours, ≤0.1 hours, ≤3 minutes, ≤1 minute, ≤30 seconds, or ≤0.1 second. In some embodiments, the hydrocarbon-containing feed and catalyst can be contacted under a hydrocarbon partial pressure of at least 20 kPa-absolute, where the hydrocarbon partial pressure is the total partial pressure of any C₂-C₁₆ alkanes and any C₈-C₁₆ alkyl aromatics in the hydrocarbon-containing feed. The catalyst can include from 0.06 wt % to 6 wt % of the Group 10 element, e.g., Pt, based on the weight of the inorganic support.

A precursor catalyst can be obtained from the at least partially deactivated catalyst. In some embodiments, the at least partially deactivated catalyst can be provided directly as the precursor catalyst. In other embodiments, the precursor catalyst can be obtained by heating the at least partially deactivated catalyst using a heating gas mixture that includes H₂O at a concentration of greater than 5 mol %, based on the total moles in the heating gas mixture to produce the precursor catalyst. In some embodiments, the heating gas mixture can be produced by combusting at least a portion of the contaminant, e.g., coke and/or residual hydrocarbon-containing feed, disposed on the at least partially deactivated catalyst with an oxidizing gas. In some embodiments, the heating gas mixture can be produced by combusting a fuel with the oxidizing gas. In other embodiments, the heating gas mixture can be produced by combusting at least a portion of the contaminant disposed on the at least partially deactivated catalyst and the fuel with the oxidizing gas. In other embodiments, the heating gas mixture that can include H₂O at a concentration of greater than 5 mol % of H₂O can be provided with the H₂O, e.g., heated air having greater than 5 mol % of H₂O. The fuel can be or can include, but is not limited to at least one of H₂, CO, and a hydrocarbon. The oxidizing gas can be or can include, but is not limited to, O₂, O₃, CO, or any mixture thereof. In some embodiments, the heating gas mixture can contact the partially deactivated catalyst for a duration <5 min, <2 min, <1 min, <30 s, <10 s, <5 s, <1 s, <0.5 s, <0.1 s.

An oxidative gas can be provided. The oxidative gas can include no greater than 5 mol % of H₂O, no greater than 4.5 mol % of H₂O, no greater than 4 mol % of H₂O, no greater than 3.5 mol % of H₂O, no greater than 3 mol % of H₂O, no greater than 2.5 mol % of H₂O, no greater than 2 mol % of H₂O, no greater than 1.7 mol % of H₂O, no greater than 1.5 mol % of H₂O, no greater than 1.3 mol % of H₂O, no greater than 1 mol % of H₂O, no greater than 0.7 mol % of H₂O, no greater than 0.5 mol % of H₂O, no greater than 0.3 mol % of H₂O, or no greater than 0.1 mol % of H₂O, based on the total moles in the oxidative gas. The precursor catalyst, whether the at least partially deactivated catalyst is provided directly as the precursor catalyst or the at least partially deactivated catalyst is heated using the heating gas mixture, can be contacted with the oxidative gas.

It has been surprisingly and unexpectedly discovered that contacting the precursor catalyst, whether the at least partially deactivated catalyst is provided directly as the precursor catalyst or the at least partially deactivated catalyst is heated using the heating gas mixture to produce the precursor catalyst, with the oxidative gas that includes no greater than 5 mol % of H₂O can significantly improve the activity and/or selectivity of the regenerated catalyst. Without wishing to be bound by theory, it is believed that an H₂O present in the oxidative gas may significantly reduce the effectiveness of Pt re-dispersion and hence the effectiveness of the regenerated catalyst.

The precursor catalyst can be contacted with the oxidative gas at an oxidizing temperature in a range from 620° C., 650° C., 675° C., 700° C., or 750° C. to 775° C., 800° C., 850° C., 900° C., 950° C., or 1,000° C. to produce an oxidized precursor catalyst. The precursor catalyst can be contacted with the oxidative gas for a duration of at least 30 seconds, at least 1 minute, at least 5 minutes, at least 7 minutes, at least 10 minutes, at least 15 minutes, at least 20 minutes, at least 25 minutes, at least 30 minutes, at least 45 minutes, at least 60 min or at least 120 minutes to produce the oxidized precursor catalyst. In some embodiments, the precursor catalyst can be contacted with the oxidative gas for a duration in a range from 30 seconds, 1 minute, 5 minutes, or 10 minutes to 30 minutes, 60 minutes, or 120 minutes to produce the oxidized precursor catalyst. In some embodiments, the precursor catalyst and the oxidative gas can be contacted with one another for a duration of ≤2 hours, ≤1 hour, ≤30 minutes, ≤10 minutes, ≤5 minutes, ≤1 min, ≤30 seconds, ≤10 seconds, ≤5 seconds, or ≤1 second to produce the oxidized precursor catalyst. For example, the precursor catalyst and oxidative gas can be contacted with one another for a duration in a range from 2 seconds to 2 hours to produce the oxidized precursor catalyst. In some embodiments, the precursor catalyst and oxidative gas can be contacted for a duration sufficient to remove ≥50 wt %, ≥75 wt %, or ≥90 wt % or ≥99 wt % of the contaminant, e.g., coke disposed on the precursor catalyst.

The precursor catalyst and oxidative gas can be contacted with one another under an oxidative gas partial pressure in a range from 5 kPa-absolute, 10 kPa-absolute, 20 kPa-absolute, 50 kPa-absolute, 100 kPa-absolute, 300 kPa-absolute, 500 kPa-absolute, 750 kPa-absolute, or 1,000 kPa-absolute to 1,500 kPa-absolute, 2,500 kPa-absolute, 4,000 kPa-absolute, 5,000 kPa-absolute, 7,000 kPa-absolute, 8,500 kPa-absolute, or 10,000 kPa-absolute to produce the oxidized precursor catalyst. In some embodiments, the oxidative gas partial pressure during contact with the precursor catalyst can be in a range from 5 kPa-absolute, 10 kPa-absolute, 20 kPa-absolute, 50 kPa-absolute, 100 kPa-absolute, 150 kPa-absolute, 200 kPa-absolute, 250 kPa-absolute, or 300 kPa-absolute to 500 kPa-absolute, 600 kPa-absolute, 700 kPa-absolute, 800 kPa-absolute, 900 kPa-absolute, or 1,000 kPa-absolute to produce the oxidized precursor catalyst.

Without wishing to be bound by theory, it is believed that at least a portion of the Group 10 element, e.g., Pt, disposed on the precursor catalyst can be agglomerated as compared to the catalyst prior to contact with the hydrocarbon-containing feed. It is believed that during contact of the precursor catalyst with the oxidative gas, when at least a portion of the contaminant on the precursor catalyst can be combusted, at least a portion of the Group 10 element can be re-dispersed about the inorganic support. Re-dispersing at least a portion of the agglomerated Group 10 element can increase the activity and improve the stability of the catalyst over many cycles.

In some embodiments, the oxidative gas can be provided at a temperature below the oxidizing temperature and the oxidative gas can be pre-heated to a temperature higher than the temperature of the precursor catalyst before contacting the precursor catalyst with the oxidative gas at the oxidizing temperature. In some embodiments, the oxidative gas can be pre-heated by using a radiant/conductive heat source, a heat exchanger, or a combination thereof. In other embodiments, the oxidative gas, the precursor catalyst, or both the oxidative gas and the precursor catalyst can be heated by using a radiant/conductive heat source, a heat exchanger, or a combination thereof. In other words, the precursor catalyst and/or the oxidative gas can be heated separately and then contacted with one another at the oxidizing temperature or heated in the presence of one another to the oxidizing temperature. In some embodiments, the radiant/conductive heat source can be or can include one or more electric heating elements.

A regenerated catalyst can be obtained from the oxidized precursor catalyst. In some embodiments, the oxidized precursor catalyst can be provided directly as the regenerated catalyst. In some embodiments, the oxidized precursor catalyst can optionally be contacted with a first stripping gas that can be free of O₂ to produce a stripped oxidized precursor catalyst and the regenerated catalyst can be obtained from the stripped oxidized precursor catalyst. The first stripping gas can be or can include, but is not limited to, CO, CO₂, N₂, a C₁-C₄ hydrocarbon, H₂O, He, Ne, Ar, or any mixture thereof. In some embodiments, the stripped oxidized precursor catalyst can be provided directly as the regenerated catalyst.

In some embodiments, at least a portion of the Group 10 element, e.g., Pt, in the oxidized precursor catalyst can be at a higher oxidized state as compared to the Group 10 element in the catalyst contacted with the hydrocarbon-containing feed and as compared to the Group 10 element in the at least partially deactivated catalyst. In some embodiments, the oxidized precursor catalyst or the stripped oxidized precursor catalyst can be contacted with a H₂-containing atmosphere to produce a reduced catalyst. In other embodiments, the oxidized precursor catalyst or the stripped oxidized precursor catalyst can be contacted with an atmosphere containing H₂, CO, CH₄, C₂H₆, C₃H₈, C₂H₄, C₃H₆, steam, or a mixture thereof to produce the reduced catalyst. In some embodiments, the atmosphere contacted with the oxidized precursor catalyst can also include an inert gas such as Ar, Ne, He, N₂, CO₂, H₂O, or a mixture thereof. In such embodiments, at least a portion of the Group 10 element in the reduced catalyst can be reduced to a lower oxidation state, e.g., the elemental state, as compared to the Group 10 element in the oxidized precursor catalyst.

In some embodiments, the oxidized precursor catalyst or the stripped oxidized precursor catalyst can be contacted with the H₂-containing atmosphere or the atmosphere containing H₂, CO, CH₄, C₂H₆, C₃H₈, C₂H₄, C₃H₆, steam, or a mixture thereof at a temperature in a range from 400° C., 450° C., 500° C., 550° C., 600° C., 620° C., 650° C., or 670° C. to 720° C., 750° C., 800° C., or 900° C. The oxidized precursor catalyst or the stripped oxidized precursor catalyst and the H₂-containing atmosphere or the atmosphere containing H₂, CO, CH₄, C₂H₆, C₃H₈, C₂H₄, C₃H₆, steam, or a mixture thereof can be contacted for a duration in a range from 0.01 seconds, 0.1 seconds, 1 second, 5 seconds, 10 seconds, 20 seconds, 30 seconds, or 1 minute to 10 minutes, 30 minutes, or 60 minutes. The oxidized precursor catalyst or the stripped oxidized precursor catalyst and the H₂-containing atmosphere or the atmosphere containing H₂, CO, CH₄, C₂H₆, C₃H₈, C₂H₄, C₃H₆, steam, or a mixture thereof can be contacted at a reducing agent partial pressure of 0.1 kPa-absolute, 1 kPa-absolute, 5 kPa-absolute, 10 kPa-absolute, 20 kPa-absolute, 50 kPa-absolute, or 100 kPa-absolute, 300 kPa-absolute, 500 kPa-absolute, 750 kPa-absolute, or 1,000 kPa-absolute to 1,500 kPa-absolute, 2,500 kPa-absolute, 4,000 kPa-absolute, 5,000 kPa-absolute, 7,000 kPa-absolute, 8,500 kPa-absolute, or 10,000 kPa-absolute, where the reducing agent includes any H₂, CO, CH₄, C₂H₆, C₃H₈, C₂H₄, C₃H₆, and steam. In other embodiments, the reducing agent partial pressure can be in a range from 0.1 kPa-absolute, 1 kPa-absolute, 5 kPa-absolute, 10 kPa-absolute, 20 kPa-absolute, 50 kPa-absolute, 100 kPa-absolute, 150 kPa-absolute, 200 kPa-absolute, 250 kPa-absolute, or 300 kPa-absolute to 500 kPa-absolute, 600 kPa-absolute, 700 kPa-absolute, 800 kPa-absolute, 900 kPa-absolute, or 1,000 kPa-absolute to produce the regenerated catalyst, where the reducing agent includes any H₂, CO, CH₄, C₂H₆, C₃H₈, C₂H₄, C₃H₆, and steam.

In some embodiments, the oxidized precursor catalyst or the stripped oxidized precursor catalyst can be contacted with the H₂-containing atmosphere at a temperature higher than a use temperature of the regenerated catalyst. In such embodiment, the reduced catalyst can be cooled to the use temperature. In some embodiments, the reduced catalyst can be cooled to the use temperature in a duration no greater than 20 minutes, no greater than 15 minutes, no greater than 10 minutes, no greater than 7 minutes, no greater than 5 minutes, no greater than 2 minutes, no greater than 1 minute, no greater than 30 seconds, no greater than 10 seconds, no greater than 5 seconds, no greater than 2 seconds, no greater than 1 second, no greater than 0.1 seconds, no greater than 0.01 seconds, or no greater than 0.001 seconds. The use temperature of the catalyst is the temperature at which the hydrocarbon-containing feed or the additional quantity of the hydrocarbon-containing feed is contacted with the catalyst or the regenerated catalyst to effect one or more of dehydrogenation, dehydroaromatization, and dehydrocyclization of at least a portion of the hydrocarbon-containing feed to produce the at least partially deactivated catalyst that can include the Group 10 element, the inorganic support, and the contaminant and the effluent that can include the one or more upgraded hydrocarbons and molecular hydrogen.

The regenerated catalyst can be obtained from the reduced catalyst. In some embodiments, the reduced catalyst can be provided directly as the regenerated catalyst. In other embodiments, the reduced catalyst can be contacted with a second stripping gas to produce the regenerated catalyst. The second stripping gas can be or can include, but is not limited to, CO, CO₂, N₂, a C₁-C₄ hydrocarbon, H₂O, He, Ne, Ar, or any mixture thereof.

At least a portion of the regenerated catalyst, new or fresh catalyst, or a mixture thereof can be contacted with the additional quantity of the hydrocarbon-containing feed within the reaction or conversion zone to produce additional effluent and additional at least partially deactivated catalyst. The cycle time from the contacting the hydrocarbon-containing feed with the catalyst to the contacting the additional quantity of the hydrocarbon-containing feed with at least a portion of the regenerated catalyst and optionally with new or fresh catalyst can be ≤5 hours, ≤4.5 hours, ≤4 hours, ≤3.5 hours, ≤3 hours, ≤2.5 hours, ≤2 hours, ≤1 hour, ≤0.5 hours, ≤0.2 hours, ≤0.1 hours, ≤0.05 hours, or ≤0.01 hours.

The first cycle begins upon contact of the catalyst with the hydrocarbon-containing feed, followed by contact with at least the oxidative gas to produce the oxidized precursor catalyst, which can be provided directly as the regenerated catalyst, or at least the oxidative gas and the optional reducing gas to produce the regenerated catalyst, and the first cycle ends upon contact of the regenerated catalyst with the additional quantity of the hydrocarbon-containing feed. If the first stripping gas and/or the second stripping gas or any other stripping gas(es) are utilized between flows of the hydrocarbon-containing feed and the oxidative gas, between the oxidative gas and the reducing gas (if used), between the oxidative gas and the additional quantity of the hydrocarbon-containing feed, and/or between the reducing gas (if used) and the additional quantity of the hydrocarbon-containing feed, the period of time such stripping gas(es) is/are utilized would be included in the period included in the cycle time. As such, the cycle time from the contacting the hydrocarbon-containing feed with the catalyst in step to the contacting the additional quantity of the hydrocarbon-containing feed with the regenerated catalyst can be ≤5 hours.

The catalyst that includes a Group 10 element, e.g., Pt, and the inorganic support can remain sufficiently active and stable after many cycles, e.g., at least 15, at least 20, at least 30, at least 40, at least 50, at least 60, at least 70, at least 100 cycles, at least 125 cycles, at least 150 cycles, at least 175 cycles, or at least 200 cycles with each cycle time lasting for ≤5 hours, ≤4 hours, ≤3 hours, ≤2 hours, ≤1 hour, ≤50 minutes, ≤45 minutes, ≤30 minutes, ≤15 minutes, ≤10 minutes, ≤5 minutes, ≤1 minute, ≤30 seconds, or ≤10 seconds. In some embodiments, the cycle time can be from 5 seconds, 30 seconds, 1 minute or 5 minutes to 10 minutes, 20 minutes, 30 minutes, 45 minutes, 50 minutes, 70 minutes, 2 hours, 3 ours, 4 hours, or 5 hours. In some embodiments, after the catalyst performance stabilizes (sometimes the first few cycles can have a relatively poor or a relatively good performance, but the performance can eventually stabilize), the process can produce a first upgraded hydrocarbon product yield, e.g., propylene when the hydrocarbon-containing feed includes propane, at an upgraded hydrocarbon selectivity, e.g., propylene, of ≥75%, ≥80%, ≥85%, or ≥90%, or ≥95% when initially contacted with the hydrocarbon-containing feed, and can have a second upgraded hydrocarbon product yield upon completion of the last cycle (at least 15 cycles total) that can be at least 90%, at least 93%, at least 95%, at least 97%, at least 98%, at least 99%, at least 99.5%, or at least 100% of the first upgraded hydrocarbon product yield at an upgraded hydrocarbon selectivity, e.g., propylene, of ≥75%, ≥80%, ≥85%, or ≥90%, or ≥95%.

In some embodiments, when the hydrocarbon-containing feed includes propane and the upgraded hydrocarbon includes propylene, contacting the hydrocarbon-containing feed with the catalyst can produce a propylene yield of at least 45%, at least 50%, at least 52%, at least 53%, at least 55%, at least 57%, at least 60%, at least 62%, at least 63%, at least 64%, at least 65%, at or at least 66% at a propylene selectivity of at least 75%, at least 80%, at least 85%, at least 90%, or at least 95% for at least 15, at least 20, at least 30, at least 40, at least 50, at least 60, at least 70, at least 100 cycles, at least 125 cycles, at least 150 cycles, at least 175 cycles, or at least 200 cycles. In other embodiments, when the hydrocarbon-containing feed includes at least 70 vol % of propane, based on a total volume of the hydrocarbon-containing feed, is contacted under a propane partial pressure of at least 20 kPa-absolute, a propylene yield of at least 45%, at least 50%, at least 52%, at least 53%, at least 55%, at least 57%, at least 60%, at least 62%, at least 63%, at least 64%, at least 65%, or at least 66% at a propylene selectivity of at least 75%, at least 80%, at least 85%, at least 90%, or at least 95% can be obtained for at least 15, at least 20, at least 30, at least 40, at least 50, at least 60, at least 70, at least 100 cycles, at least 125 cycles, at least 150 cycles, at least 175 cycles, or at least 200 cycles. It is believed that the propylene yield can be further increased to at least 67%, at least 68%, at least 70%, at least 72%, at least 75%, at least 77%, at least 80%, or at least 82% at a propylene selectivity of at least 75%, at least 80%, at least 85%, at least 90%, or at least 95% for at least 15 cycles, at least 20, at least 30, at least 40, at least 50, at least 60, at least 70, at least 100 cycles, at least 125 cycles, at least 150 cycles, at least 175 cycles, or at least 200 cycles by further optimizing the composition of the support and/or adjusting one or more process conditions. In some embodiments, the propylene yield can be obtained when the catalyst is contacted with the hydrocarbon feed at a temperature of at least 620° C., at least 630° C., at least 640° C., at least 650° C., at least 655° C., at least 660° C., at least 670° C., at least 680° C., at least 690° C., at least 700° C., or at least 750° C. for at least 15, at least 20, at least 30, at least 40, at least 50, at least 60, at least 70, at least 100 cycles, at least 125 cycles, at least 150 cycles, at least 175 cycles, or at least 200 cycles.

Systems suitable for carrying out the processes disclosed herein can include systems that are well-known in the art such as the fixed bed reactors disclosed in WO Publication No. WO2017078894; the fluidized riser reactors and/or downer reactors disclosed in U.S. Pat. Nos. 3,888,762; 7,102,050; 7,195,741; 7,122,160; and 8,653,317; and U.S. Patent Application Publication Nos. 2004/0082824; 2008/0194891; and the reverse flow reactors disclosed in U.S. Pat. No. 8,754,276; U.S. Patent Application Publication No. 2015/0065767; and WO Publication No. WO2013169461.

### Catalyst

The catalyst can include 0.06 wt %, 0.07 wt %, 0.08 wt %, 0.09 wt %, 0.1 wt %, 0.2 wt %, 0.5 wt %, or 1 wt % to 2 wt %, 3 wt %, 4 wt %, 5 wt %, or 6 wt % of the Group 10 element, based on the weight of the inorganic support. In some embodiments, the catalyst can include >0.06 wt %, >0.08 wt %, >0.1 wt %, >0.13 wt %, >0.15 wt %, >0.17 wt %, >0.2 wt %, >0.2 wt %, >0.23, >0.25 wt %, >0.27 wt %, or >0.3 wt % and <0.5 wt %, <1 wt %, <2 wt %, <3 wt %, <4 wt %, <5 wt %, or <6 wt % of the Group 10 element, based on the weight of the inorganic support. In some embodiments, the Group 10 element can be or can include Ni, Pd, Pt, a combination thereof, or a mixture thereof. In at least one embodiment, the Group 10 element can be or can include Pt. In some embodiments, an active component of the regenerated catalyst that can be capable of effecting one or more of dehydrogenation, dehydroaromatization, and dehydrocyclization of the hydrocarbon-containing feed that includes one or more of C₂-C₁₆ linear or branched alkanes, or one or more of C₄-C₁₆ cyclic alkanes, one or more C₈-C₁₆ alkyl aromatics, or a mixture thereof can include the group 10 element.

The inorganic support can be or can include, but is not limited to, one or more Group 4 elements, a combination thereof, or a mixture thereof. In some embodiments, the Group 4 element can be present in its elemental form. In other embodiments, the Group 4 element can be present in the form of a compound. For example, the Group 4 element can be present as an oxide, a phosphate, a halide, a halate, a sulfate, a sulfide, a borate, a nitride, a carbide, an aluminate, an aluminosilicate, a silicate, a carbonate, metaphosphate, a selenide, a tungstate, a molybdate, a chromite, a chromate, a dichromate, or a silicide. In some embodiments, a mixture of any two or more compounds that include the Group 4 element can be present in different forms. For example, a first compound can be an oxide and a second compound can be an aluminate where the first compound and the second compound include the same or different Group 4 element, with respect to one another.

In some embodiments, at least a portion of the Group 4 element can be in the form of ZrO₂. In some embodiments, at least a portion of the Group 4 element can be in the form of ZrO₂ or a mixed oxide that includes ZrO₂. In some embodiments, when the Group 4 element is in the form of a mixed oxide that includes ZrO₂, the other oxide can be or can include, but is not limited to, Al₂O₃, SiO₂, TiO₂, MgO, CeO₂, or any mixture thereof. In some embodiments, the Group 4 element can be or can include, but is not limited to, one or more of the following compounds: ZrO₂, ZrC, ZrN, ZrSiO₄, CaZrO₃, Ca₇ZrAl₆O₁₈, CaTiO₃, TiO₂, TiC, TiN, TiSiO₄, CaTiO₃, HfO₂, HfC, HfN, HfSiO₄, HfZrO₃, Ca₇HfAl₆O₁₈, CeZrO₄, sulfated zirconia, tungstated zirconia, zirconia alumina, magnesia stabilized zirconia, magnesium zirconium oxide, cerium zirconium oxide, combinations thereof, and mixtures thereof.

The inorganic support can include ≥0.5 wt %, ≥1 wt %, ≥2 wt %, ≥3 wt %, ≥4 wt %, ≥5 wt %, ≥10 wt %, or ≥20 wt %, ≥40 wt %, ≥80 wt %, or ≥90 wt % of the Group 4 element, based on the weight of the inorganic support. In some embodiments, the inorganic support can include the Group 4 element in a range from 0.5 wt %, 3 wt %, 5 wt %, or 10 wt % to 30 wt %, 50 wt %, 70 wt %, or 90 wt %, based on the weight of the inorganic support. In some embodiments, a molar ratio of the Group 4 element to the Group 10 element can be in a range from 0.18, 0.3, 0.5, 1, 10, 50, 100, or 200 to 300, 400, 500, 600, 700, or 810.

In some embodiments, the inorganic support can also include, but is not limited to, at least one metal element and/or at least one metalloid element selected from Groups other than Group 4 and Group 10 and/or at least one compound thereof, where the at least one metal element and/or at least one metalloid element is not Li, Na, K, Rb, Cs, Sn, Ga, Zn, Ge, In, Re, Ag, Au, or Cu. If the support also includes the at least one metal element and/or metalloid element selected from Groups other than Group 4 and Group 10, where the at least one metal element and/or at least one metalloid element is not Li, Na, K, Rb, Cs, Sn, Ga, Zn, Ge, In, Re, Ag, Au, or Cu, the compound can be present in the support as an oxide, a phosphate, a halide, a halate, a sulfate, a sulfide, a borate, a nitride, a carbide, an aluminate, an aluminosilicate, a silicate, a carbonate, metaphosphate, a selenide, a tungstate, a molybdate, a chromite, a chromate, a dichromate, or a silicide. In some embodiments, suitable compounds that include the metal element and/or metalloid element selected from Groups other than Group 4 and Group 10, where the at least one metal element and/or at least one metalloid element is not Li, Na, K, Rb, Cs, Sn, Ga, Zn, Ge, In, Re, Ag, Au, or Cu, can be or can include, but are not limited to, one or more of the following: B₂O₃, AlBO₃, Al₂O₃, SiO₂, SiC, Si₃N₄, an aluminosilicate, zinc aluminate, ZnO, VO, V₂O₃, VO₂, V₂O₅, GasOt, InuOv, Mn₂O₃, Mn₃O₄, MnO, one or more molybdenum oxides, one or more tungsten oxides, one or more zeolites, where s, t, u, and v are positive numbers and mixtures and combinations thereof.

In some embodiments, the at least one metal element and/or at least one metalloid element selected from Groups other than Group 4 and Group 10 and/or at least one compound thereof, where the at least one metal element and/or at least one metalloid element is not Li, Na, K, Rb, Cs, Sn, Ga, Zn, Ge, In, Re, Ag, Au, or Cu can be or can include, but is not limited to, one or more elements having an atomic number of 57 to 71. In such embodiments, the catalyst can include 0.01 wt %, 0.05 wt %, 0.1 wt %, 0.3 wt %, 0.5 wt %, 0.7 wt %, or 0.9 wt % to 1 wt %, 2 wt %, 3 wt %, 4 wt %, 5 wt %, 6 wt %, 7 wt %, 8 wt % 9 wt %, or 10 wt % of a total amount of the one or more elements having an atomic number of 57 to 71, based on the weight of the inorganic support. In some embodiments, when the catalyst includes the element having an atomic number of 57 to 71, a molar ratio of the element having an atomic number of 57 to 71 to the Group 10 element can be in a range from 0.19, 0.5, 1, 10, 50, 100, or 150 to 200, 250, 300, 350, 400, or 438. In some embodiments, when the catalyst includes two or more Group 4 elements and/or elements having an atomic number of 57-71, a molar ratio of a combined amount of any Group 4 element and any element having an atomic number of 57-71 to the Group 10 element can be in a range from 0.18, 0.5, 1, 10, 50, 100, 300, 450, 600, 800, 1,000, 1,200, 1,500, 1,700, or 2,000 to 3,000, 3,500, 4,000, 4,500, 5,000, 5,500, 6,000, 6,500, 7,000, 7,500, 8,000, 8,500, 9,000, or 9,500.

In some embodiments, the one or more elements having an atomic number of 57 to 71 can be or can include, but is not limited to, La, Ce, Pr, a combination thereof, or a mixture thereof. In some embodiments, the one or more elements having an atomic number of 57 to 71 can be present as an oxide, a phosphate, a halide, a halate, a sulfate, a sulfide, a borate, a nitride, a carbide, an aluminate, an aluminosilicate, a silicate, a carbonate, metaphosphate, a selenide, a tungstate, a molybdate, a chromite, a chromate, a dichromate, or a silicide.

In some embodiments, the inorganic support can also include one or more promoters disposed thereon. The promoter can be or can include, but is not limited to, Sn, Ga, Zn, Ge, In, Re, Ag, Au, Cu, a combination thereof, or a mixture thereof. In some embodiments, the promoter can be associated with the Group 10 element, e.g., Pt. For example, the promoter and the Group 10 element disposed on the inorganic support can form Group 10 element-promoter clusters that can be dispersed on the inorganic support. The promoter, if present, can improve the selectivity/activity/longevity of the catalyst for a given upgraded hydrocarbon. In some embodiments, the addition of the promoter can improve the propylene selectivity of the catalyst when the hydrocarbon-containing feed includes propane. The catalyst can include the promoter in an amount of 0.01 wt %, 0.1 wt %, 0.2 wt %, 0.3 wt %, 0.4 wt %, 0.5 wt %, 0.6 wt %, 0.7 wt %, 0.8 wt %, 0.9 wt %, or 1 wt % to 3 wt %, 5 wt %, 7 wt %, or 10 wt %, based on the weight of the inorganic support.

In some embodiments, the inorganic support can also include one or more alkali metal elements disposed thereon. The alkali metal element, if present, can be or can include, but is not limited to, Li, Na, K, Rb, Cs, a combination thereof, or a mixture thereof. In at least some embodiments, the alkali metal element ca be or can include K and/or Cs. The alkali metal element, if present, can improve the selectivity of the catalyst for a given upgraded hydrocarbon. The catalyst can include the alkali metal element in an amount 0.01 wt %, 0.1 wt %, 0.2 wt %, 0.3 wt %, 0.4 wt %, 0.5 wt %, 0.6 wt %, 0.7 wt %, 0.8 wt %, 0.9 wt %, or 1 wt % to 2 wt %, 3 wt %, 4 wt %, or 5 wt %, based on the weight of the inorganic support. In some embodiments, suitable catalyst can include those described in U.S. Pat. Nos. 6,989,346; 7,087,802; and 8,680,005 and U.S. Patent Application Publication No. 2007/009929.

The preparation of the inorganic support can be accomplished via any known process. For simplicity and ease of description, the preparation of a suitable inorganic support that includes a mixed oxide of ZrO₂ and SiO₂ inorganic support will be described in more detail. Catalyst synthesis techniques are well-known and the following description is for illustrative purposes and not to be considered as limiting the synthesis of the inorganic support or the catalyst. In some embodiments, to make the ZrO₂/SiO₂ mixed oxide inorganic support, ZrO₂ and SiO₂ can be mixed together, e.g., ball-milled, followed by calcination. In some embodiments, to make the ZrO₂/SiO₂ mixed oxide inorganic support, ZrO₂ and SiO₂ can be mixed together, e.g., ball-milled, slurried, followed by spray drying and calcination. In another embodiment, a Zr-containing and a Si-containing precursor can be dissolved in H₂O, stirred until dry (with heat or a precipitation agent optionally applied), followed by calcination. In another embodiment, the Si-containing precursor can be dissolved in H₂O and the solution can be impregnated onto an existing inorganic support, e.g., a ZrO₂ inorganic support, that can be dried and calcined. In another embodiment, Si from a Si-containing precursor can be loaded onto an existing ZrO₂ inorganic support through liquid phase adsorption, followed by liquid-solid separation, drying and calcination. In another embodiment, Si from a Si-containing precursor can be loaded onto an existing ZrO₂ inorganic support through gas phase adsorption such as chemical vapor deposition, followed by calcination.

Group 10 metal(s) and any promoter and/or any alkali metal element and/or any at least one metal element and/or at least one metalloid element selected from Groups other than Group 4 and Group 10 and/or at least one compound thereof, where the at least one metal element and/or at least one metalloid element is not Li, Na, K, Rb, Cs, Sn, Ga, Zn, Ge, In, Re, Ag, Au, or Cu can be loaded onto the mixed oxide inorganic support by any known technique. For example, one or more Group 10 element precursors, e.g., chloroplatinic acid, tetramineplatinum nitrate, and/or tetramineplatinum hydroxide, one or more promoter precursors (if used), e.g., a salt such as SnCl₂, SnCl₄ and/or AgNO₃, and one or more alkali metal element precursors (if used), e.g., KNO₃, KCl, and/or NaCl, can be dissolved in water. The solution can be impregnated onto the inorganic support, followed by drying and calcination. In some embodiments, the Group 10 element precursor and optionally the promoter precursor and/or optionally the alkali metal element precursor and/or optionally the at least one metal element and/or at least one metalloid element that is not Li, Na, K, Rb, Cs, Sn, Ga, Zn, Ge, In, Re, Ag, Au, or Cu can be loaded onto the inorganic support at the same time, or separately in a sequence separated by one or more drying and/or calcination steps. In other embodiments, the Group 10 element and, optionally the promoter and/or alkali metal element and/or the at least one metal element and/or at least one metalloid element selected from Groups other than Group 4 and Group 10 and/or at least one compound thereof, where the at least one metal element and/or at least one metalloid element is not Li, Na, K, Rb, Cs, Sn, Ga, Zn, Ge, In, Re, Ag, Au, or Cu, can be loaded onto the inorganic support by chemical vapor deposition, where the precursors are volatilized and deposited onto the inorganic support, followed by calcination. In other embodiments, the Group 10 element precursor and, optionally, the promoter precursor and/or alkali metal precursor and/or the at least one metal element and/or at least one metalloid element selected from Groups other than Group 4 and Group 10 element and/or at least one compound thereof, where the at least one metal element and/or at least one metalloid element is not Li, Na, K, Rb, Cs, Sn, Ga, Zn, Ge, In, Re, Ag, Au, or Cu, can be loaded onto the inorganic support through ion adsorption, followed by liquid-solid separation, drying and calcination. Optionally, the catalyst can also be synthesized using a one-pot synthesis method where the precursors of the inorganic support, the Group 10 metal(s) and any promoter and/or any alkali metal element and/or any at least one metal element and/or at least one metalloid element selected from Groups other than Group 4 and Group 10 and/or at least one compound thereof, where the at least one metal element and/or at least one metalloid element is not Li, Na, K, Rb, Cs, Sn, Ga, Zn, Ge, In, Re, Ag, Au, or Cu can all be mixed together, dry or wet, with or without any other additives to aid the synthesis, followed by drying and calcination.

Suitable processes that can be used to prepare the catalysts disclosed herein can include the processes described in U.S. Pat. Nos. 6,989,346; 7,087,802; and 8,680,005 and U.S. Patent Application Publication No. 2007/009929.

The as-synthesized catalyst, when examined under scanning electron microscope or transmission electron microscope, can appear as either primary particles, as agglomerates of primary particles, as aggregated primary particles, or a combination thereof. The primary particles in the as-synthesized catalyst, when examined under scanning electron microscope or transmission electron microscope, can have an average particle size, e.g., a diameter when spherical, in a range from 0.2 nm, 0.5 nm, 1 nm, 5 nm, 10 nm, 25 nm, 30 nm, 40 nm 50 nm, 60 nm, 70 nm, 80 nm, 90 nm, 100 nm, 150 nm, 200 nm, 250 nm, 300 nm, 350 nm, 400 nm, 450 nm, or 500 nm to 1 μm, 10 μm, 25 μm, 50 μm, 100 μm, 150 μm, 200 μm, 250 μm, 300 μm, 400 μm, or 500 μm. In some embodiments, the catalyst particles can have an average cross-sectional length of 0.2 nm to 500 μm, 0.5 nm to 300 μm, 1 nm to 200 μm, 2 nm to 100 μm, or 2 nm to 500 nm as measured by a transmission electron microscope.

The catalyst can have a surface area in a range from 0.1 m²/g, 1 m²/g, 10 m²/g, or 100 m²/g to 500 m²/g, 800 m²/g, 1,000 m²/g, or 1,500 m²/g. The surface area of the catalyst can be measured according to the Brunauer-Emmett-Teller (BET) method using adsorption-desorption of nitrogen (temperature of liquid nitrogen, 77 K) with a Micromeritics 3flex instrument after degassing of the powders for 4 hrs at 350° C. More information regarding the method can be found, for example, in “Characterization of Porous Solids and Powders: Surface Area, Pore Size and Density,” S. Lowell et al., Springer, 2004.

In some embodiments, the inorganic support can be extruded or otherwise formed into any desired monolithic structure and the Group 10 element and any optional promoter and/or alkali metal element and/or other component can be disposed thereon. Suitable monolithic structures can be or can include, but are not limited to, structures having a plurality of substantially parallel internal passages such as those in the form of a ceramic honeycomb. In some embodiments, the support can be in the form of beads, spheres, rings, toroidal shapes, irregular shapes, rods, cylinders, flakes, films, cubes, polygonal geometric shapes, sheets, fibers, coils, helices, meshes, sintered porous masses, granules, pellets, tablets, powders, particulates, extrudates, cloth or web form materials, honeycomb matrix monolith, including in comminuted or crushed forms, and the Group 10 element and any optional promoter and/or alkali metal element can be disposed thereon.

The as-synthesized catalyst can be formulated into one or more appropriate forms for different short cycle (≤5 hours) hydrocarbon upgrading processes. Alternatively, the support can be formulated into appropriate forms for different short cycle hydrocarbon upgrading processes, before the addition of the Group 10 element and, any optional promoter and/or alkali metal element. During formulation, one or more binders and/or additives can be added to the catalyst/support or catalyst/support precursors to improve the chemical/physical properties of the catalyst. Spray-dried catalyst particles having an average cross-sectional diameter in a range from 40 μm to 100 μm are typically used in an FCC type fluid-bed reactor. To make spray-dried catalyst, it is preferred that the support/catalyst or support/catalyst precursor be made into a slurry with binder/additive in the slurry before spray-drying and calcination. In some embodiments, the spray-dried catalyst can be in the form of particles and the morphology of the particles can be largely spherical so that the particles are suitable to run in a fluid bed reactor. In some embodiments, the catalyst particles can have a size and density that is consistent with a Geldart A or Geldart B definition of a fluidizable solid.

### Hydrocarbon Upgrading Process

Returning to the hydrocarbon upgrading process, the hydrocarbon-containing feed and the catalyst and/or at least a portion of the regenerated catalyst can be contacted with one another within any suitable environment such as one or more reaction or conversion zones disposed within one or more reactors to produce the effluent and the at least partially deactivated catalyst. In some embodiments, the reaction or conversion zone can be disposed or otherwise located within one or more fixed bed reactors, one or more fluidized or moving bed reactors, one or more reverse flow reactors, or any combination thereof.

The hydrocarbon-containing feed and catalyst and/or at least a portion of the regenerated catalyst can be contacted at a temperature in a range from 300° C., 350° C., 400° C., 450° C., 500° C., 550° C., 600° C., 620° C., 650° C., 660° C., 670° C., 680° C., 690° C., or 700° C. to 725° C., 750° C., 760° C., 780° C., 800° C., 825° C., 850° C., 875° C., or 900° C. In some embodiments, the hydrocarbon-containing feed and catalyst and/or at least a portion of the regenerated catalyst can be contacted at a temperature of at least 620° C., at least 650° C., at least 660° C., at least 670° C., at least 680° C., at least 690° C., or at least 700° C. to 725° C., 750° C., 760° C., 780° C., 800° C., 825° C., 850° C., 875° C., or 900° C. The hydrocarbon-containing feed can be introduced into the reaction or conversion zone and contacted with the catalyst and/or at least a portion of the regenerated catalyst therein for a time period of ≤3 hours, ≤2.5 hours, ≤2 hours, ≤1.5 hours, ≤1 hour, ≤45 minutes, ≤30 minutes, ≤20 minutes, ≤10 minutes, ≤5 minutes, ≤1 minute, ≤30 seconds, ≤10 seconds, ≤5 seconds, or ≤1 second or ≤0.5 second. In some embodiments, the hydrocarbon-containing feed can be contacted with the catalyst and/or at least a portion of the regenerated catalyst for a time period in a range from 0.1 seconds, 0.5 seconds, 0.7 seconds, 1 second, 30 second, 1 minute, 5 minutes, or 10 minutes to 30 minutes, 50 minutes, 70 minutes, 1.5 hours, 2 hours, or 3 hours.

The hydrocarbon-containing feed and the catalyst and/or at least a portion of the regenerated catalyst can be contacted under a hydrocarbon partial pressure of at least 20 kPa-absolute, where the hydrocarbon partial pressure is the total partial pressure of any C₂-C₁₆ alkanes and any C₈-C₁₆ alkyl aromatics in the hydrocarbon-containing feed. In some embodiments, the hydrocarbon partial pressure during contact of the hydrocarbon-containing feed and the catalyst and/or at least a portion of the regenerated catalyst can be in a range from 20 kPa-absolute, 50 kPa-absolute, 100 kPa-absolute, at least 150 kPa, at least 200 kPa 300 kPa-absolute, 500 kPa-absolute, 750 kPa-absolute, or 1,000 kPa-absolute to 1,500 kPa-absolute, 2,500 kPa-absolute, 4,000 kPa-absolute, 5,000 kPa-absolute, 7,000 kPa-absolute, 8,500 kPa-absolute, or 10,000 kPa-absolute, where the hydrocarbon partial pressure is the total partial pressure of any C₂-C₁₆ alkanes and any C₈-C₁₆ alkyl aromatics in the hydrocarbon-containing feed. In other embodiments, the hydrocarbon partial pressure during contact of the hydrocarbon-containing feed and the catalyst and/or at least a portion of the regenerated catalyst can be in a range from 20 kPa-absolute, 50 kPa-absolute, 100 kPa-absolute, 150 kPa-absolute, 200 kPa-absolute, 250 kPa-absolute, or 300 kPa-absolute to 500 kPa-absolute, 600 kPa-absolute, 700 kPa-absolute, 800 kPa-absolute, 900 kPa-absolute, or 1,000 kPa-absolute, where the hydrocarbon partial pressure is the total partial pressure of any C₂-C₁₆ alkanes and any C₈-C₁₆ alkyl aromatics in the hydrocarbon-containing feed.

In some embodiments, the hydrocarbon-containing feed can include at least 60 vol %, at least 65 vol %, at least 70 vol %, at least 75 vol %, at least 80 vol %, at least 85 vol %, at least 90 vol %, at least 95 vol %, or at least 99 vol % of a single C₂-C₁₆ alkane, e.g., propane, based on a total volume of the hydrocarbon-containing feed. The hydrocarbon-containing feed and catalyst and/or at least a portion of the regenerated catalyst can be contacted under a single C₂-C₁₆ alkane, e.g., propane, pressure of at least 20 kPa-absolute, at least 50 kPa-absolute, at least 100 kPa-absolute, at least 150 kPa-absolute, at least 250 kPa-absolute, at least 300 kPa-absolute, at least 400 kPa-absolute, at least 500 kPa-absolute, or at least 1,000 kPa-absolute.

The hydrocarbon-containing feed can be contacted with the catalyst and/or at least a portion of the regenerated catalyst within the reaction or conversion zone at any weight hourly space velocity (WHSV) effective for carrying out the upgrading process. In some embodiments, the WHSV can be 0.01 hr⁻¹, 0.1 hr⁻¹, 1 hr⁻¹, 2 hr⁻¹, 5 hr⁻¹, 10 hr⁻¹, 20 hr⁻¹, 30 hr⁻¹, or 50 hr⁻¹ to 100 hr⁻¹, 250 hr⁻¹, 500 hr⁻¹, or 1,000 hr⁻¹. In some embodiments, when the hydrocarbon upgrading process includes a fluidized or otherwise moving catalyst and/or moving regenerated catalyst, a ratio of the catalyst circulation mass flow rate to a combined amount of any C₂-C₁₆ alkanes and any C₈-C₁₆ alkyl aromatics mass flow rate can be in a range from 1, 3, 5, 10, 15, 20, 25, 30, or 40 to 50, 60, 70, 80, 90, 100, 110, 125, or 150 on a weight to weight basis.

When the activity of the at least partially deactivated catalyst decreases below a desired minimum amount, the at least partially deactivated catalyst or at least a portion thereof can be subjected to the regeneration process described above to produce the regenerated catalyst. Regeneration of the at least partially deactivated catalyst can occur within the reaction or conversion zone or within a combustion zone that is separate and apart from the reaction or conversion zone, depending on the particular reactor configuration, to produce a regenerated catalyst. For example, regeneration of the catalyst can occur within the reaction or conversion zone when a fixed bed or reverse flow reactor is used, or within a separate combustion zone that can be separate and apart from the reaction or conversion zone when a fluidized bed reactor or other circulating or fluidized type reactor is used. Similarly, the optional reduction step can also occur within the reaction or conversion zone, within the combustion zone, and/or within a separate reduction zone. Accordingly, the hydrocarbon containing feed can be contacted with the catalyst to effect one or more of dehydrogenation, dehydroaromatization, and dehydrocyclization of at least a portion of the hydrocarbon-containing feed to produce a coked catalyst and a first effluent that includes the one or more upgraded hydrocarbons and molecular hydrogen in a cyclic type process such as those commonly employed in fixed bed and reverse flow reactors and/or a continuous type process commonly employed in fluidized bed reactors. The separation of the effluent that includes the upgraded hydrocarbon and molecular hydrogen from the coked catalyst, if needed, can be accomplished via one or more separators such as a cyclone separator. As noted above, the oxidative gas can be or can include, but is not limited to, O₂, O₃, CO₂, or a mixture thereof and can include no greater than 5 mol % of H₂O. In some embodiments, an amount of oxidative gas in excess of that needed to combust 100% of the contaminant, e.g., coke, disposed on the catalyst can be used to increase the rate of contaminant removal from the catalyst, so that the time needed for removal of the contaminant can be reduced and lead to an increased yield in the upgraded product produced within a given period of time.

### Hydrocarbon-Containing Feed

The C₂-C₁₆ alkanes can be or can include, but are not limited to, ethane, propane, n-butane, isobutane, n-pentane, isopentane, n-hexane, 2-methylpentane, 3-methylpentane, 2,2-dimethylbutane, n-heptane, 2-methylhexane, 2,2,3-trimethylbutane, cyclopentane, cyclohexane, methylcyclopentane, ethylcyclopentane, n-propylcyclopentane, 1,3-dimethylcyclohexane, or a mixture thereof. For example, the hydrocarbon-containing feed can include propane, which can be dehydrogenated to produce propylene, and/or isobutane, which can be dehydrogenated to produce isobutylene. In another example, the hydrocarbon-containing feed can include liquid petroleum gas (LP gas), which can be in the gaseous phase when contacted with the catalyst. In some embodiments, the hydrocarbon in the hydrocarbon-containing feed can be composed of substantially a single alkane such as propane. In some embodiments, the hydrocarbon-containing feed can include ≥50 mol %, ≥75 mol %, ≥95 mol %, ≥98 mol %, or ≥99 mol % of a single C₂-C₁₆ alkane, e.g., propane, based on a total weight of all hydrocarbons in the hydrocarbon-containing feed. In some embodiments, the hydrocarbon-containing feed can include at least 50 vol %, at least 55 vol %, at least 60 vol %, at least 65 vol %, at least 70 vol %, at least 75 vol %, at least 80 vol %, at least 85 vol %, at least 90 vol %, at least 95 vol %, at least 97 vol %, or at least 99 vol % of a single C₂-C₁₆ alkane, e.g., propane, based on a total volume of the hydrocarbon-containing feed.

The C₈-C₁₆ alkyl aromatics can be or can include, but are not limited to, ethylbenzene, propylbenzenes, butylbenzenes, one or more ethyl toluenes, or a mixture thereof. In some embodiments, the hydrocarbon-containing feed can include ≥50 mol %, ≥75 mol %, ≥95 mol %, ≥98 mol %, or ≥99 mol % of a single C₈-C₁₆ alkyl aromatic, e.g., ethylbenzene, based on a total weight of all hydrocarbons in the hydrocarbon-containing feed. In some embodiments, the ethylbenzene can be dehydrogenated to produce styrene. As such, in some embodiments, the processes disclosed herein can include propane dehydrogenation, butane dehydrogenation, isobutane dehydrogenation, pentane dehydrogenation, pentane dehydrocyclization to cyclopentadiene, naphtha reforming, ethylbenzene dehydrogenation, ethyltoluenes dehydrogenation, and the like.

In some embodiments, the hydrocarbon-containing feed can be diluted, e.g., with one or more diluents such as one or more inert gases. Suitable inert gases can be or can include, but are not limited to, Ar, Ne, He, N₂, CO₂, CH₄, or a mixture thereof. If the hydrocarbon containing-feed includes a diluent, the hydrocarbon-containing feed can include 0.1 vol %, 0.5 vol %, 1 vol %, or 2 vol % to 3 vol %, 8 vol %, 16 vol %, or 32 vol % of the diluent, based on a total volume of any C₂-C₁₆ alkanes and any C₈-C₁₆ alkyl aromatics in the hydrocarbon-containing feed.

In some embodiments, the hydrocarbon-containing feed can also include H₂. In some embodiments, when the hydrocarbon-containing feed includes H₂, a molar ratio of the H₂ to a combined amount of any C₂-C₁₆ alkane and any C₈-C₁₆ alkyl aromatic can be in a range from 0.1, 0.3, 0.5, 0.7, or 1 to 2, 3, 4, 5, 6, 7, 8, 9, or 10.

In some embodiments, the hydrocarbon-containing feed can be substantially free of any steam, e.g., <0.1 vol % of steam, based on a total volume of any C₂-C₁₆ alkanes and any C₈-C₁₆ alkyl aromatics in the hydrocarbon-containing feed. In other embodiments, the hydrocarbon-containing feed can include steam. For example, the hydrocarbon-containing feed can include 0.1 vol %, 0.3 vol %, 0.5 vol %, 0.7 vol %, 1 vol %, 3 vol %, or 5 vol % to 10 vol %, 15 vol %, 20 vol %, 25 vol %, 30 vol %, 35 vol %, 40 vol %, 45 vol %, or 50 vol % of steam, based on a total volume of any C₂-C₁₆ alkanes and any C₈-C₁₆ alkyl aromatics in the hydrocarbon-containing feed. In other embodiments, the hydrocarbon-containing feed can include ≤50 vol %, ≤45 vol %, ≤40 vol %, ≤35 vol %, ≤30 vol %, ≤25 vol %, ≤20 vol %, or ≤15 vol % of steam, based on a total volume of any C₂-C₁₆ alkanes and any C₈-C₁₆ alkyl aromatics in the hydrocarbon-containing feed. In other embodiments, the hydrocarbon-containing feed can include at least 1 vol %, at least 3 vol %, at least 5 vol %, at least 10 vol %, at least 15 vol %, at least 20 vol %, at least 25 vol %, or at least 30 vol % of steam, based on a total volume of any C₂-C₁₆ alkanes and any C₈-C₁₆ alkyl aromatics in the hydrocarbon-containing feed.

In some embodiments, the hydrocarbon-containing feed can include sulfur. For example, the hydrocarbon-containing feed can include sulfur in a range from 0.5 ppm, 1 ppm, 5 ppm, 10 ppm, 20 ppm 30 ppm, 40 ppm, 50 ppm, 60 ppm, 70 ppm, or 80 ppm to 100 ppm, 150 ppm, 200 ppm, 300 ppm, 400 ppm, or 500 ppm. In other embodiments, the hydrocarbon-containing feed can include sulfur in a range from 1 ppm to 10 ppm, 10 ppm to 20 ppm, 20 ppm to 50 ppm, 50 ppm to 100 ppm, or 100 ppm to 500 ppm. The sulfur, if present in the hydrocarbon-containing feed, can be or can include, but is not limited to, H₂S, dimethyl disulfide, as one or more mercaptans, or any mixture thereof.

The hydrocarbon feed can be substantially free or free of molecular oxygen. In some embodiments, the hydrocarbon feed can include ≤5 mol %, ≤3 mol %, or ≤1 mol % of molecular oxygen (O₂). It is believed that providing a hydrocarbon feed substantially-free of molecular oxygen substantially prevents oxidative reactions that would otherwise consume at least a portion of the alkane and/or the alkyl aromatic in the hydrocarbon feed.

### Recovery and Use of the Upgraded Hydrocarbons

The upgraded hydrocarbon can include at least one upgraded hydrocarbon, e.g., an olefin, water, unreacted hydrocarbons, molecular hydrogen, etc. The upgraded hydrocarbon can be recovered or otherwise obtained via any convenient process, e.g., by one or more conventional processes. One such process can include cooling and/or compressing the effluent to condense at least a portion of any water and any heavy hydrocarbon that may be present, leaving the olefin and any unreacted alkane or alkyl aromatic primarily in the vapor phase. Olefin and unreacted alkane or alkyl aromatic hydrocarbons can then be removed from the reaction product in one or more separator devices such as a distillation column, an adsorptive separation device, a membrane separation device, a cryogenic separation device, etc. For example, one or more splitters or distillation columns can be used to separate the dehydrogenated product from the unreacted hydrocarbon feed.

In some embodiments, a recovered olefin, e.g., propylene, can be used for producing polymer, e.g., recovered propylene can be polymerized to produce polymer having segments or units derived from the recovered propylene such as polypropylene, ethylene-propylene copolymer, etc. Recovered isobutene can be used, e.g., for producing one or more of: anoxygenate such as methyl tert-butyl ether, fuel additives such as diisobutene, synthetic elastomeric polymer such as butyl rubber, etc.

### EXAMPLES

The foregoing discussion can be further described with reference to the following non-limiting examples. Catalyst 1 and a comparative catalyst 1 were prepared according to the following procedures.
Catalyst 1: The catalyst was prepared according to the following procedure: A solution of 0.036 g of SnCl₂·2H₂O and 0.0236 g of H₂PtCl₆·6H₂O in 18.375 ml (14.5 g) of ethanol was poured over 3 g of a ZrO₂ doped with 5% SiO₂ (Saint-Gobain) while the mixture was stirred with a magnetic stirring bar. The excess solution was evaporated overnight. The composition was dried at 100° C. for 15 hours and calcined at 560° C. for 3 hours to produce calcined solid particles. A solution of 0.0232 g of CsNO₃, 0.0408 g of KNO₃ and 0.295 g of La(NO)₃·6H₂O in 11.25 ml of H₂O was then poured over the calcined solid particles while the mixture was stirred with a magnetic stirring bar. The supernatant solution was evaporated at a temperature of 60° C. to 90° C. over a few days. The final solid was dried at 110° C. for 6 hours and calcined at 800° C. for 12 hours to produce re-calcined catalyst particles.
Comparative catalyst 1: In a graduated cylinder, SnCl₂ (0.048 g) (Aldrich), chloroplatinic acid, 8% solution, (0.79 g) (Aldrich), and remainder HCl (1.2 M) (Acculute) were combined to make a dark solution of 5.6 mL. The solution was added to theta-alumina (10 g) and stirred for 15 minutes. The catalyst was allowed to rest for 1 hr. The catalyst was placed in a muffle furnace and ramped at 3° C./min to 120° C., held for 2 hours at 120° C., and then the catalyst was ramped at 3° C./min to 550° C., which was maintained for 2 hours, all in air. The catalyst was then cooled to room temperature.

In a graduated cylinder, KNO₃ (0.258 g) (Aldrich) was dissolved in deionized water to yield 5.6 mL of solution. The solution was added to the Pt—Sn catalyst and stirred for 15 minutes. The catalyst was allowed to rest for 1 hr. The catalyst was placed in a muffle furnace and ramped at 3° C./min to 120° C., held for 2 hours at 120° C., and then the catalyst was ramped at 3° C./min to 550° C., which was maintained for 2 hours, all in air. The catalyst was then cooled to room temperature. The final product contained nominally 0.3 wt % Pt, 0.3 wt % Sn, and 1.0 wt % K.

### Examples Using the Catalysts Described Above.

Fixed bed experiments were conducted at approximately 100 kPa-absolute. A gas chromatograph (GC) was used to measure the composition of the reactor effluents. The concentrations of each component in the reactor effluents were then used to calculate the C₃H₆ yield and selectivity. The C₃H₆ yield and selectivity, as reported in these examples, were calculated on the carbon mole basis.

In each example, a certain amount of the catalyst “Mcat” was mixed with an appropriate amount of quartz diluent and loaded into a quartz reactor. The amount of diluent was determined so that the catalyst bed (catalyst+diluent) overlaps with the isothermal zone of the quartz reactor and the catalyst bed is largely isothermal during operation. The dead volume of the reactor was filled with quartz chips/rods.

The concentration of each component in the reactor effluent was used to calculate the C₃H₆ yield and selectivity. The C₃H₆ yield and the selectivity at the beginning of tᵣₓₙ and at the end of tᵣₓₙ is denoted as Yᵢₙᵢ, Y_{end}, Sᵢₙᵢ, and S_{end}, respectively, and reported as percentages in the data tables below.
Example 1—Regeneration Temperature/Duration for Catalyst 1. 1. The system was flushed with an inert gas while the reaction zone was heated to a regeneration temperature T_{regen}. 1. An oxygen containing gas (Ogas) at a flow rate (F_{regen}) was passed through the by-pass of the reaction zone, while an inert gas was flown through the reaction zone. 3. The oxygen containing gas was then passed through the reaction zone for a certain period of time (t_{regen}) to regenerate the catalyst. 4. After t_{regen}, an inert gas was passed through the reaction zone and the temperature within the reaction zone was changed from T_{regen} to a reduction temperature (T_{red}). 5. A H₂ containing gas (Hgas) at a flow rate (F_{red}) was passed through the by-pass of the reaction zone for a certain period of time, while an inert gas was passed through the reaction zone. This was then followed by passing the H₂ containing gas through the reaction zone at T_{red} for a certain period of time (t_{red}). 6. The system was flushed with an inert. During this process, the temperature of the reaction zone was changed from T_{red} to a reaction temperature of Tᵣₓₙ° C. 7. A hydrocarbon-containing (HCgas) feed that included 81 vol % C₃H₈, 9 vol % inert (Ar or Kr) and 10 vol % steam at a flow rate (Fᵣₓₙ) was passed through the by-pass of the reaction zone for a certain period of time, while an inert gas was passed through the reaction zone. The hydrocarbon-containing feed was then passed through the reaction zone at Tᵣₓₙ° C. for 10 min. GC sampling of the reaction effluent started as soon as the feed was switched from the by-pass of the reaction zone to the reaction zone. Tables 1-3 show the effect of regeneration duration/temperature to the PDH performance of catalyst.

TABLE 1

| | | |
|---|---|---|
| Catalyst | 28 | 28 |

| M_{cat} (g) | | | 0.3 | 0.3 |
|---|---|---|---|---|
| Fᵣₓₙ (sccm) | | | 9.4 | 9.4 |
| Hgas | | | 10% H₂ | 10% H₂ |
| | | | 90% Ar | 90% Ar |
| F_{red} (sccm) | | | 46.6 | 46.6 |
| T_{red} (° C.) | | | 670 | 670 |
| t_{red} (min) | | | 1 | 1 |
| Ogas | | | Dry air | Dry air |
| F_{regen} (sccm) | | | 83.9 | 83.9 |
| T_{regen} (° C.) | | | 670 | 670 |
| t_{regen} (min) | | | 30 | 15 |
| Tᵣₓₙ (° C.) | | | 650 | 650 |
| | Performance | Yᵢₙᵢ | 55.6 | 44.1 |
| | | Y_{end} | 44.4 | 34.9 |
| | | Sᵢₙᵢ | 94.7 | 95.3 |
| | | S_{end} | 95.6 | 95.1 |

TABLE 2

| Catalyst | | 28 | 28 | 28 | 28 | 28 | 28 | 28 |
|---|---|---|---|---|---|---|---|---|
| M_{cat} (g) | | 0.3 | 0.3 | 0.3 | 0.3 | 0.3 | 0.3 | 0.3 |
| Fᵣₓₙ (sccm) | | 9.4 | 9.4 | 9.4 | 9.4 | 9.4 | 9.4 | 9.4 |
| Hgas | | 10% H₂ | 10% H₂ | 10% H₂ | 10% H₂ | 10% H₂ | 10% H₂ | 10% H₂ |
| | | 90% Ar | 90% Ar | 90% Ar | 90% Ar | 90% Ar | 90% Ar | 90% Ar |
| F_{red} (sccm) | | 46.6 | 46.6 | 46.6 | 46.6 | 46.6 | 46.6 | 46.6 |
| T_{red} (° C.) | | 620 | 620 | 620 | 620 | 620 | 620 | 620 |
| t_{red} (min) | | 1 | 1 | 1 | 1 | 1 | 1 | 1 |
| Ogas | | Dry | Dry | Dry | Dry | Dry | Dry | Dry |
| | | air | air | air | air | air | air | air |
| F_{regen} (sccm) | | 83.9 | 83.9 | 83.9 | 83.9 | 83.9 | 83.9 | 83.9 |
| T_{regen} (° C.) | | 620 | 620 | 620 | 680 | 800 | 800 | 800 |
| t_{regen} (min) | | 45 | 30 | 10 | 30 | 30 | 10 | 5 |
| Tᵣₓₙ (° C.) | | 620 | 620 | 620 | 620 | 620 | 620 | 620 |
| Performance | Yᵢₙᵢ | 48.2 | 47.8 | 25.3 | 48.1 | 41.4 | 38.8 | 32.9 |
| | Y_{end} | 44.1 | 44.4 | 20.7 | 43.7 | 29.5 | 28.6 | 25.4 |
| | Sᵢₙᵢ | 95.9 | 96.4 | 96.4 | 95.5 | 96.9 | 97 | 96.2 |
| | S_{end} | 96.7 | 97.4 | 96.6 | 97.7 | 97.3 | 97.4 | 95.9 |

TABLE 3

| Catalyst | | 28 | 28 | 28 | 28 | 28 |
|---|---|---|---|---|---|---|
| M_{cat} (g) | | 0.3 | 0.3 | 0.3 | 0.3 | 0.3 |
| Fᵣₓₙ (sccm) | | 9.4 | 9.4 | 9.4 | 9.4 | 9.4 |
| Hgas | | 10% | 10% | 10% | 10% | 10% |
| | | H₂ | H₂ | H₂ | H₂ | H₂ |
| | | 90% | 90% | 90% | 90% | 90% |
| | | Ar | Ar | Ar | Ar | Ar |
| F_{red} (sccm) | | 46.6 | 46.6 | 46.6 | 46.6 | 46.6 |
| T_{red} (° C.) | | 620 | 650 | 670 | 670 | 700 |
| t_{red} (min) | | 1 | 1 | 1 | 1 | 1 |
| Ogas | | Dry | Dry | Dry | Dry | Dry |
| | | air | air | air | air | air |
| F_{regen} (sccm) | | 83.9 | 83.9 | 83.9 | 83.9 | 83.9 |
| T_{regen} (° C.) | | 620 | 650 | 670 | 670 | 700 |
| t_{regen} (min) | | 30 | 30 | 30 | 30 | 30 |
| Tᵣₓₙ (° C.) | | 650 | 650 | 650 | 670 | 670 |
| Performance | Yᵢₙᵢ | 55 | 53.4 | 54.1 | 56.3 | 56 |
| | Y_{end} | 46.8 | 45.5 | 45.7 | 43.9 | 41.8 |
| | Sᵢₙᵢ | 94 | 94.4 | 94.3 | 92 | 92.4 |
| | S_{end} | 95.5 | 94.9 | 95.5 | 92.7 | 92.7 |

Example 2—Effect of Catalyst Reduction and Steam Co-feed for Catalyst 1. 1. The system was flushed with an inert gas while the reaction zone was heated to a regeneration temperature T_{regen}. 2. An oxygen containing gas (Ogas) at a flow rate (F_{regen}) was passed through the by-pass of the reaction zone, while an inert gas was passed through the reaction zone. 3. The oxygen containing gas was then passed through the reaction zone for a certain period of time (t_{regen}) to regenerate the catalyst. 4. After t_{regen}, an inert gas was passed through the reaction zone and the temperature within the reaction zone was changed from T_{regen} to a reduction temperature (T_{red}). 5. A H₂ containing gas (Hgas) at a flow rate (F_{red}) was passed through the by-pass of the reaction zone for a certain period of time, while an inert gas was passed through the reaction zone. This was followed by flowing the H₂ containing gas through the reaction zone at T_{red} for a certain period of time (t_{red}). 6. The system was flushed with an inert gas. During this process, the temperature of the reaction zone was changed from T_{red} to a reaction temperature of Tᵣₓₙ° C. 7. A hydrocarbon-containing (HCgas) at a flow rate (Fᵣₓₙ) was passed through the by-pass of the reaction zone for a certain period of time, while an inert gas was through the reaction zone. The hydrocarbon-containing feed was then passed through the reaction zone at Tᵣₓₙ° C. for 10 min. GC sampling of the reaction effluent started as soon as the feed was switched from the by-pass of the reaction zone to the reaction zone. Table 4 shows that both catalyst is most active for PDH when there is catalyst reduction and steam co-feed.

TABLE 4

| Catalyst | | 28 | 28 | 28 | 28 |
|---|---|---|---|---|---|
| M_{cat} (g) | | 0.3 | 0.3 | 0.3 | 0.3 |
| Fᵣₓₙ (sccm) | | 9.4 | 8.5 | 9.4 | 8.5 |
| Hgas | | 10% | 10% | 10% | 10% |
| | | H₂ | H₂ | H₂ | H₂ |
| | | 90% | 90% | 90% | 90% |
| | | Ar | Ar | Ar | Ar |
| F_{red} (sccm) | | 46.6 | 46.6 | 46.6 | 46.6 |
| T_{red} (° C.) | | 670 | NA | 670 | NA |
| t_{red} (min) | | 1 | NA | 1 | NA |
| Ogas | | 90% | 90% | 90% | 90% |
| | | Air | Air | Air | Air |
| | | 10% | 10% | 10% | 10% |
| | | H₂O | H₂O | H₂O | H₂O |
| | | Then | Then | Then | Then |
| | | Dry | Dry | Dry | Dry |
| | | air | air | air | air |
| F_{regen} (sccm) | | 93.2 Then 83.9 | 93.2 Then 83.9 | 93.2 Then 83.9 | 93.2 Then 83.9 |
| T_{regen} (° C.) | | 670 | 670 | 670 | 670 |
| t_{regen} (min) | | 1 Then 30 | 1 Then 30 | 1 Then 30 | 1 Then 30 |
| Tᵣₓₙ (° C.) | | 650 | 650 | 650 | 650 |
| HCgas | | 81% | 90% | 90% | 81% |
| | | C₃H₈, | C₃H₈ | C₃H₈ | C₃H₈, |
| | | 9% | 10% | 10% | 9% |
| | | Ar | Ar | Ar | Ar |
| | | 10% | | | 10% |
| | | Steam | | | Steam |
| Performance | Yᵢₙᵢ | 52.1 | 14.1 | 19.4 | 23.1 |
| | Y_{end} | 44.3 | 6.2 | 9.5 | 19.1 |
| | Sᵢₙᵢ | 94.4 | 90 | 92.5 | 91 |
| | S_{end} | 95.1 | 79.1 | 85.4 | 91.4 |

Example 3—Effect of Steam during Regeneration Catalyst 1. 1. The system was flushed with an inert gas while the reaction zone was heated to a regeneration temperature T_{regen}. 2. An oxygen containing gas (Ogas) at a flow rate (F_{regen}) was passed through the by-pass of the reaction zone, while an inert gas was passed through the reaction zone. 3. The oxygen containing gas was then passed through the reaction zone for a certain period of time (t_{regen}) to regenerate the catalyst. 4. After t_{regen}, an inert gas was passed through the reaction zone and the temperature within the reaction zone was changed from T_{regen} to a reduction temperature (T_{red}). 5. The system was flushed with an inert gas. 6. A H₂ containing gas (Hgas) at a flow rate (F_{red}) was passed through the by-pass of the reaction zone for a certain period of time, while an inert gas was passed through the reaction zone. This was then followed by passing the H₂ containing gas through the reaction zone at T_{red} for a certain period of time (t_{red}). 7. The system was flushed with an inert gas. During this process, the temperature of the reaction zone was changed from T_{red} to a reaction temperature of 650° C. 8. A hydrocarbon-containing (HCgas) feed that included 81 vol % C₃H₈, 9 vol % inert (Ar or Kr) and 10 vol % steam at a flow rate (Fᵣₓₙ) was passed through the by-pass of the reaction zone for a certain period of time, while an inert gas was passed through the reaction zone. The hydrocarbon-containing feed was then passed through the reaction zone at 650° C. for 10 min. GC sampling of the reaction effluent started as soon as the feed was switched from the by-pass of the reaction zone to the reaction zone. The above process steps were repeated in cycles until stable performance was obtained. Table 5 shows that the presence of more than 10 vol % of steam in air during regeneration yielded an even more deactivated catalyst after regeneration. On the other hand, if the moist air is switched to dry air after 1 min of regeneration, the catalyst was effectively regenerated.

TABLE 5

| | | | |
|---|---|---|---|
| Catalyst | 28 | 28 | 28 |

| M_{cat} (g) | | 0.3 | 0.3 | 0.3 |
|---|---|---|---|---|
| Fᵣₓₙ (sccm) | | 9.4 | 9.4 | 9.4 |
| Hgas | | 10% H₂ | 10% H₂ | 10% H₂ |
| | | 90% Ar | 90% Ar | 90% Ar |
| F_{red} (sccm) | | 46.6 | 46.6 | 46.6 |
| T_{red} (° C.) | | 670 | 670 | 670 |
| t_{red} (min) | | 1 | 1 | 1 |
| Ogas | | Dry air | 90% Air | 90% Air |
| | | | 10% H₂O | 10% H₂O |
| | | | | Then |
| | | | | Dry air |
| F_{regen} (sccm) | | 83.9 | 93.2 | 93.2 Then 83.9 |
| T_{regen} (° C.) | | 670 | 670 | 670 |
| t_{regen} (min) | | 30 | 30 | 1 Then 30 |
| Performance | Yᵢₙᵢ | 55.6 | 38.9 | 55.6 |
| | Y_{end} | 44.4 | 31.9 | 48.3 |
| | Sᵢₙᵢ | 94.7 | 94.8 | 94.4 |
| | S_{end} | 95.6 | 94.8 | 95.6 |

Example 4—Effect of H₂ Reduction Duration for Catalyst 1. 1. The system was flushed with an inert gas while the reaction zone was heated to a regeneration temperature of 700° C. 2. An oxygen containing gas (Ogas) at a flow rate (F_{regen}) was passed through the by-pass of the reaction zone, while an inert gas was passed through the reaction zone. 3. The oxygen containing gas was then passed through the reaction zone for a certain period of time (t_{regen}) to regenerate the catalyst. 4. The system was flushed with an inert gas. During this process, the temperature of the reaction zone was kept at 700° C. 5. A H₂ containing gas (Hgas) at a flow rate (F_{red}) was passed through the by-pass of the reaction zone for a certain period of time, while an inert gas was passed through the reaction zone. This was then followed by flowing the H₂ containing gas through the reaction zone at 700° C. for a certain period of time (t_{red}). 6. He gas was passed through the reaction zone. During this process, the temperature of the reaction zone was reduced from 700° C. to a reaction temperature of 650° C. 7. A hydrocarbon-containing (HCgas) feed that included 81 vol % C₃H₈, 9 vol % inert (Ar or Kr) and 10 vol % steam at a flow rate (Fᵣₓₙ) was passed through the by-pass of the reaction zone for a certain period of time, while an inert gas was passed through the reaction zone. The hydrocarbon-containing feed was then passed through the reaction zone at 650° C. for 10 min. GC sampling of the reaction effluent started as soon as the feed was switched from the by-pass of the reaction zone to the reaction zone. The above process steps were repeated in cycles until stable performance was obtained. Table 6 shows the effect of reduction duration to the performance of the catalyst.

TABLE 6

| Catalyst | | 28 | 28 | 28 | 28 |
|---|---|---|---|---|---|
| M_{cat} (g) | | 0.3 | 0.3 | 0.3 | 0.3 |
| Fᵣₓₙ (sccm) | | 9.4 | 9.4 | 9.4 | 9.4 |
| Hgas | | 10% | 10% | 10% | 10% |
| | | H₂ | H₂ | H₂ | H₂ |
| | | 90% | 90% | 90% | 90% |
| | | Ar | Ar | Ar | Ar |
| F_{red} (sccm) | | 46.6 | 46.6 | 46.6 | 46.6 |
| t_{red} (min) | | 4 | 1 | 0.6 | 0.2 |
| Ogas | | 90% | 90% | 90% | 90% |
| | | Air | Air | Air | Air |
| | | 10% | 10% | 10% | 10% |
| | | H₂O | H₂O | H₂O | H₂O |
| | | Then | Then | Then | Then |
| | | Dry | Dry | Dry | Dry |
| | | air | air | air | air |
| F_{regen} (sccm) | | 93.2 Then 83.9 | 93.2 Then 83.9 | 93.2 Then 83.9 | 93.2 Then 83.9 |
| t_{regen} (min) | | 1 Then 15 | 1 Then 15 | 1 Then 15 | 1 Then 15 |
| Performance | Yᵢₙᵢ | 53.1 | 52.8 | 52.2 | 50.6 |
| | Y_{end} | 43.4 | 43.3 | 42.1 | 41.6 |
| | Sᵢₙᵢ | 94.6 | 94.6 | 94.5 | 94.4 |
| | S_{end} | 95.6 | 95.6 | 95.5 | 95.4 |

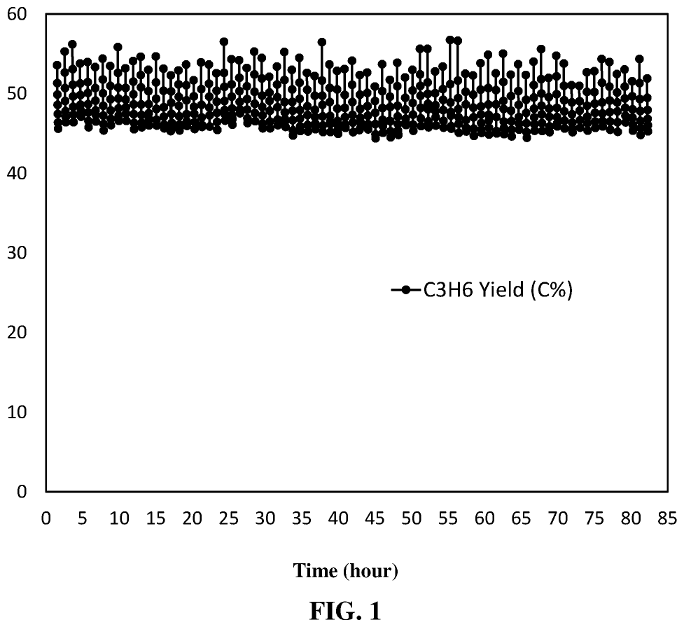

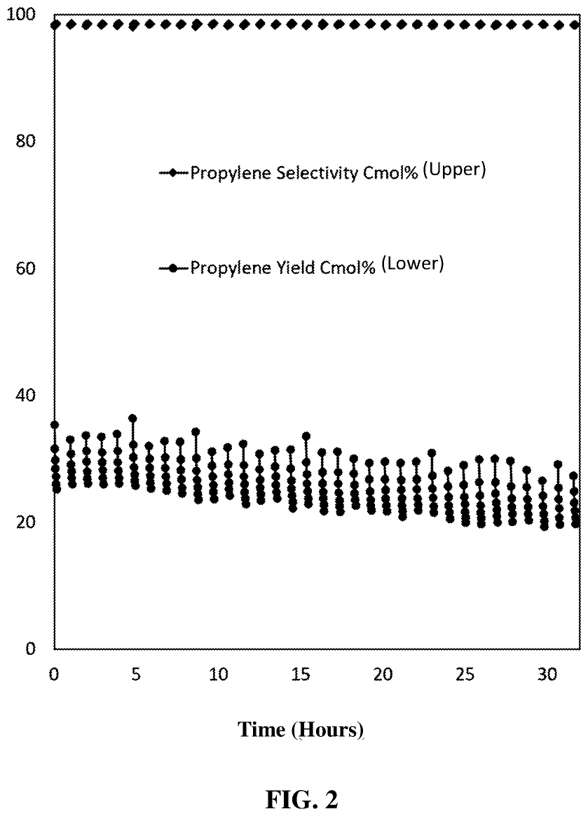

Example 5—Catalyst Life of Catalyst 1. 1. To test the catalyst life of catalyst 1, 0.3 g of catalyst 1 was mixed with an appropriate amount of quartz diluent and loaded into a quartz reactor. The amount of diluent was determined so that the catalyst bed (catalyst+diluent) overlapped with the isothermal zone of the quartz reactor and the catalyst bed was largely isothermal during operation. The dead volume of the reactor was filled with quartz chips/rods. 1. The system was flushed with an inert gas while the reaction zone was heated to a regeneration temperature 670 C. 2. A gas that contained 90% Air and 10% H₂O at a flow rate of 93.2 sccm was passed through the by-pass of the reaction zone, while an inert gas was passed through the reaction zone. 3. The gas that contained the air and H₂O was then flown through the reaction zone for 1 min to regenerate the catalyst. 4. Steam was discontinued, 83.9 sccm of dry air was flown through the reaction zone for 30 min to further regenerate the catalyst. 5. An inert gas was flown through the reaction zone and the temperature within the reaction zone was maintained at 670° C. 6. A gas that contained 10% H₂ and 90% Ar at a flow rate of 46.6 sccm was passed through the by-pass of the reaction zone for a certain period of time, while an inert gas was passed through the reaction zone. This was then followed by flowing the H₂ containing gas through the reaction zone at 670° C. for 1 min. 7. The system was flushed with an inert gas. During this process, the temperature of the reaction zone was changed from 670° C. to a reaction temperature of 650° C. 8. A hydrocarbon-containing feed that included 81 vol % C₃H₈, 9 vol % inert (Ar or Kr) and 10 vol % steam at a flow rate of 9.4 sccm was passed through the by-pass of the reaction zone for a certain period of time, while an inert gas was passed through the reaction zone. The hydrocarbon-containing feed was then passed through the reaction zone at 650° C. for 10 min. GC sampling of the reaction effluent started as soon as the feed was switched from the by-pass of the reaction zone to the reaction zone. The above process steps were repeated in cycles until stable performance was obtained. FIG. 1 shows that the performance of the catalyst for PDH was stable after 75+ cycles.
Comparative Example 1: 1. The system was flushed with an inert gas while the reaction zone was heated to an oxidation temperature of Tₒₓᵢ. 2. An oxygen containing gas (Ogas) at a flow rate (Fₒₓᵢ) was passed through the by-pass of the reaction zone, while an inert was passed through the reaction zone. 3. The oxygen containing gas was then passed through the reaction zone for a certain period of time (tₒₓᵢ) to oxidize the catalyst. 4. The system was flushed with an inert gas. During this process, the temperature of the reaction zone was cooled down to 620° C. 5. A H₂ containing gas (Hgas) at a flow rate (F_{red}) was passed through the by-pass of the reaction zone for a certain period of time, while an inert was passed through the reaction zone. This was then followed by flowing the H₂ containing gas through the reaction zone at 620° C. for a certain period of time (t_{red}). 6. The reaction zone was passed with an inert gas. During this process, the temperature of the reaction zone was maintained at 620° C. 7. A hydrocarbon-containing (HCgas) feed that included 90 vol % C₃H₈, 10 vol % inert (Ar or Kr) at a flow rate (Fᵣₓₙ) was passed through the by-pass of the reaction zone for a certain period of time, while an inert was passed through the reaction zone. The hydrocarbon-containing feed was then passed through the reaction zone at 620° C. for 10 min. GC sampling of the reaction effluent started as soon as the feed was switched from the by-pass of the reaction zone to the reaction zone. Table 7 shows further details of the testing conditions of the comparative example. FIG. 2 shows that the performance of comparative catalyst 1 kept on deactivating, even though the oxidation temperature (620° C.) was much lower than the other examples.

TABLE 7

| Catalyst | Comparative catalyst 1 |
|---|---|
| M_{cat} (g) | 0.3 |
| Fᵣₓₙ (sccm) | 15.8 |
| Hgas | 10% H₂ 90% Ar |
| F_{red} (sccm) | 46.6 |
| t_{red} (min) | 1 |
| Ogas | Dry air |
| Fₒₓᵢ (sccm) | 83.9 |
| Tₒₓᵢ (° C.) | 620 |
| tₒₓᵢ (min) | 30 |

### LISTING OF EMBODIMENTS

This disclosure further includes the following non-limiting embodiments.
A1. A process for regenerating an at least partially deactivated catalyst comprising a Group 10 element, an inorganic support, and a contaminant, wherein the Group 10 element has a concentration in the range from 0.06 wt % to 6 wt %, based on the weight of the inorganic support, and the process comprises: (I) obtaining a precursor catalyst from the at least partially deactivated catalyst; (II) providing an oxidative gas comprising no greater than 5 mol % of H₂O, based on the total moles in the oxidative gas; (III) contacting the precursor catalyst at an oxidizing temperature in a range from 620° C. to 1,000° C. with the oxidative gas for a duration of at least 30 seconds, preferably at least 1 minute, preferably at least 5 minutes, to produce an oxidized precursor catalyst; and (IV) obtaining a regenerated catalyst from the oxidized precursor catalyst.
A2. The process of A1, wherein the Group 10 element comprises Pt, and wherein the inorganic support comprises at least 0.5 wt % of a Group 4 element, based on the weight of the inorganic support.
A3. The process of A2, wherein the inorganic support comprises at least 0.5 wt % of Zr.
A4. The process of A2 or A3, wherein at least a portion of the Group 4 element is in the form of ZrO₂.
A5. The process of any one of A1 to A4, wherein the at least partially deactivated catalyst further comprises up to 10 wt % of a promoter, based on the weight of the inorganic support, and wherein the promoter comprises one or more of the following elements: Sn, Ga, Zn, Ge, In, Re, Ag, Au, Cu, a combination thereof, or a mixture thereof.
A6. The process of any one of A1 to A5, wherein the at least partially deactivated catalyst further comprises up to 5 wt % an alkali metal element disposed on the inorganic support, and wherein the alkali metal element comprises at least one of: Li, Na, K, Rb, and Cs.
A7. The process of any one of A1 to A6, wherein an active component of the regenerated catalyst that is capable of effecting one or more of dehydrogenation, dehydroaromatization, and dehydrocyclization of a hydrocarbon-containing feed comprising one or more of C₂-C₁₆ linear or branched alkanes, or one or more of C₄-C₁₆ cyclic alkanes, one or more C₈-C₁₆ alkyl aromatics, or a mixture thereof comprises the Group 10 element.
A8. The process of any one of A1 to A7, wherein step (I) comprises: heating the at least partially deactivated catalyst using a heating gas mixture comprising H₂O at a concentration of greater than 5 mol %, based on the total moles in the heating gas mixture to produce the precursor catalyst.
A9. The process of A8, wherein the heating gas mixture is produced by combusting a fuel with an oxidizing gas, and wherein the fuel comprises at least one of H₂, CO, and a hydrocarbon, and the oxidizing gas comprises O₂.
A10. The process of any one of A1 to A7, wherein, in step (I), the at least partially deactivated catalyst is provided directly as the precursor catalyst.
A11. The process of any one of A1 to A10, wherein step (II) comprises: (IIa) providing the oxidative gas at a temperature below the oxidizing temperature; and (IIb) pre-heating the oxidative gas to a temperature higher than the temperature of the precursor catalyst before the contacting in step (III).
A12. The process of any one of A1 to A11, further comprising: (V) heating the oxidative gas or the precursor catalyst during step (III) by using a radiant heat source, a heat exchanger, or a combination thereof.
A13. The process of any one of A1 to A12, wherein step (IV) comprises: (IVa) contacting the oxidized precursor catalyst with a first stripping gas free of O₂ to produce a stripped oxidized precursor catalyst; and (IVb) obtaining the regenerated catalyst from the stripped oxidized precursor catalyst.
A14. The process of any one of A1 to A13, wherein step (IV) comprises: (IVc) contacting the oxidized precursor catalyst or the stripped oxidized precursor catalyst with a H₂-containing atmosphere to produce a reduced catalyst; and (IVd) obtaining the regenerated catalyst from the reduced catalyst.
A15. The process of A14, wherein step (IVd) comprises: (IVd-1) contacting the reduced catalyst with a second stripping gas to produce the regenerated catalyst.
A16. The process of A14 or A15, wherein step (IVc) is carried out at a temperature of the oxidized precursor catalyst higher than a use temperature of the regenerated catalyst, and step (IVd) further comprises: (IVd-2) cooling the reduced catalyst or the regenerated catalyst to the use temperature in a duration no greater than 10 minutes, no greater than 5 minutes, no greater than 1 minute, no greater than 30 seconds, no greater than 10 seconds, no greater than 5 seconds, no greater than 1 second, no greater than 0.5 seconds, no greater than 0.1 seconds, no greater than 0.01 seconds, or no greater than 0.001 seconds.
A17. A dehydrogenation process using the regenerated catalyst produced by a process of any one of A1 to A16, the dehydrogenation process comprising: (VI) contacting a hydrocarbon-containing feed with the regenerated catalyst to effect one or more of dehydrogenation, dehydroaromatization, and dehydrocyclization of at least a portion of the hydrocarbon-containing feed to produce the at least partially deactivated catalyst comprising the Group 10 element, the inorganic support, and the contaminant and an effluent comprising one or more upgraded hydrocarbons and molecular hydrogen, wherein the hydrocarbon feed comprises one or more of C₂-C₁₆ linear or branched alkanes, one or more of C₄-C₁₆ cyclic alkanes, one or more of C₈-C₁₆ alkyl aromatic hydrocarbons, or a mixture thereof; and (VII) repeating steps (I) through (IV), wherein, in step (III) additional oxidized precursor catalyst is produced, and wherein, in step (IV), additional regenerated catalyst is obtained from the additional oxidized precursor catalyst; and (VIII) contacting an additional quantity of the hydrocarbon-containing feed with at least a portion of the additional regenerated catalyst to produce additional at least partially deactivated catalyst and additional effluent.
A18. The dehydrogenation process of A17, wherein a cycle time from the contacting the hydrocarbon-containing feed with the regenerated catalyst in step (VI) to the contacting the additional quantity of the hydrocarbon-containing feed with the additional regenerated catalyst in step (VIII) is ≤5 hours.
B1. A process for upgrading a hydrocarbon, comprising: (I) contacting a hydrocarbon-containing feed with a catalyst comprising a Group 10 element and an inorganic support to effect one or more of dehydrogenation, dehydroaromatization, and dehydrocyclization of at least a portion of the hydrocarbon-containing feed to produce an at least partially deactivated catalyst comprising the Group 10 element, the inorganic support, and a contaminant and an effluent comprising one or more upgraded hydrocarbons and molecular hydrogen, wherein: the hydrocarbon-containing feed comprises one or more of C₂-C₁₆ linear or branched alkanes, or one or more of C₄-C₁₆ cyclic alkanes, one or more C₈-C₁₆ alkyl aromatics, or a mixture thereof; the Group 10 element has a concentration in the range from 0.06 wt % to 6 wt %, based on the weight of the inorganic support; the hydrocarbon-containing feed and the catalyst are contacted at a temperature in a range from 300° C. to 900° C.; and the one or more upgraded hydrocarbons comprise at least one of a dehydrogenated hydrocarbon, a dehydroaromatized hydrocarbon, and a dehydrocyclized hydrocarbon; (II) obtaining a precursor catalyst from the at least partially deactivated catalyst; (III) providing an oxidative gas comprising no greater than 2 mol % of H₂O, based on the total moles in the oxidative gas; (IV) contacting the precursor catalyst at an oxidizing temperature in a range from 620° C. to 1,000° C. with the oxidative gas for a duration of at least 30 seconds, preferably at least 1 minute, preferably at least 5 minutes, to produce an oxidized precursor catalyst; (V) obtaining a regenerated catalyst from the oxidized precursor catalyst; and (VI) contacting an additional quantity of the hydrocarbon-containing feed with at least a portion of the regenerated catalyst to produce additional at least partially deactivated catalyst and additional effluent.
B2. The process of B1, wherein: the Group 10 element comprises Pt, the inorganic support comprises at least 0.5 wt % of a Group 4 element, based on the weight of the inorganic support, the catalyst optionally further comprises up to 10 wt % of a promoter, based on the weight of the inorganic support, wherein the promoter, if present, comprises one or more of the following elements: Sn, Ga, Zn, Ge, In, Re, Ag, Au, Cu, a combination thereof, or a mixture thereof, the catalyst optionally further comprises up to 5 wt % an alkali metal element, and the alkali metal element, if present, comprises at least one of: Li, Na, K, Rb, and Cs.
B3. The process of B1 or B2, wherein step (II) comprises: heating the at least partially deactivated catalyst using a heating gas mixture comprising H₂O at a concentration of greater than 5 mol %, based on the total moles in the heating gas mixture to produce the precursor catalyst.
B4. The process of B3, wherein the heating gas mixture is produced by combusting a fuel with an oxidizing gas, and wherein the fuel comprises at least one of H₂, CO, and a hydrocarbon, and the oxidizing gas comprises O₂.
B5. The process of any one of B1 to B4, wherein, in step (II), the at least partially deactivated catalyst is provided directly as the precursor catalyst.
B6. The process of any one of B1 to B5, wherein step (III) comprises: (IIIa) providing the oxidative gas at a temperature below the oxidizing temperature; and (IIIb) pre-heating the oxidative gas to a temperature higher than the temperature of the precursor catalyst before the contacting in step (IV).
B7. The process of any one of B1 to B6, further comprising: (VI) heating the oxidative gas or the precursor catalyst during step (IV) by using a radiant heat source, a heat exchanger, or a combination thereof.
B8. The process of any one of B1 to B7, wherein a cycle time from the contacting the hydrocarbon-containing feed with the catalyst in step (I) to the contacting the additional quantity of the hydrocarbon-containing feed with the regenerated catalyst in step (VI) is <5 hours.
B9. The process of any one of B2 to B8, wherein the Group 4 element comprises Zr.
B10. The process of B9, wherein the Zr is in the form of ZrO₂. Various terms have been defined above.

To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1. A process for regenerating an at least partially deactivated catalyst comprising a Group 10 element, an inorganic support, and a contaminant, wherein the Group 10 element has a concentration in the range from 0.06 wt % to 6 wt %, based on the weight of the inorganic support, and the process comprises:
(I) heating the at least partially deactivated catalyst using a heating gas mixture comprising H₂O at a concentration of greater than 5 mol %, based on the total moles in the heating gas mixture to produce a precursor catalyst;
(II) providing an oxidative gas comprising no greater than 5 mol % of H₂O, based on the total moles in the oxidative gas;
(III) contacting the precursor catalyst at an oxidizing temperature in a range from 620° C. to 1,000° C. with the oxidative gas for a duration of at least 30 seconds, to produce an oxidized precursor catalyst; and
(IV) obtaining a regenerated catalyst from the oxidized precursor catalyst.

2. The process of claim 1, wherein the heating gas mixture is produced by combusting a fuel with an oxidizing gas.

3. The process of claim 2, wherein the fuel comprises at least one of H₂, CO, and a hydrocarbon, and the oxidizing gas comprises 02.

4. The process of claim 1, wherein the Group 10 element comprises Pt.

5. The process of claim 1, wherein the inorganic support comprises at least 0.5 wt % of a Group 4 element, based on the weight of the inorganic support.

6. The process of claim 5, wherein the inorganic support comprises at least 0.5 wt % of Zr.

7. The process of claim 5, wherein at least a portion of the Group 4 element is in the form of ZrO₂.

8. The process of claim 1, wherein the at least partially deactivated catalyst further comprises one or more elements having an atomic number of 57 to 71.

9. The process of claim 8, wherein the at least partially deactivated catalyst comprises 0.01 wt % to 10 wt % of a total amount of the one or more elements having an atomic number of 57 to 71, based on the weight of the inorganic support.

10. The process of claim 1, wherein the at least partially deactivated catalyst further comprises up to 10 wt % of a promoter, based on the weight of the inorganic support, and wherein the promoter comprises one or more of the following elements: Sn, Ga, Zn, Ge, In, Re, Ag, Au, Cu, a combination thereof, or a mixture thereof.

11. The process of claim 1, wherein the at least partially deactivated catalyst further comprises up to 5 wt % of an alkali metal element, based on the weight of the inorganic support, and wherein the alkali metal element comprises at least one of: Li, Na, K, Rb, and Cs.

12. The process of claim 1, wherein an active component of the regenerated catalyst that is capable of effecting one or more of dehydrogenation, dehydroaromatization, and dehydrocyclization of a hydrocarbon-containing feed comprising one or more of C₂-C₁₆ linear or branched alkanes, or one or more of C₄-C₁₆ cyclic alkanes, one or more C₈-C₁₆ alkyl aromatics, or a mixture thereof comprises the Group 10 element.

13. The process of claim 1, wherein step (II) comprises:
(IIa) providing the oxidative gas at a temperature below the oxidizing temperature; and
(IIb) pre-heating the oxidative gas to a temperature higher than the temperature of the precursor catalyst before the contacting in step (III).

14. The process of claim 1, further comprising:
(V) heating the oxidative gas, the precursor catalyst, or both, during step (III) by using a radiant/conductive heat source, a heat exchanger, or a combination thereof.

15. The process of claim 1, wherein step (IV) comprises:
(IVa) contacting the oxidized precursor catalyst with a first stripping gas free of O₂ to produce a stripped oxidized precursor catalyst; and
(IVb) obtaining the regenerated catalyst from the stripped oxidized precursor catalyst.

16. The process of claim 1, wherein step (IV) comprises:
(IVc) contacting the oxidized precursor catalyst or the stripped oxidized precursor catalyst with a H₂-containing atmosphere to produce a reduced catalyst; and
(IVd) obtaining the regenerated catalyst from the reduced catalyst.

17. The process of claim 16, wherein step (IVd) comprises:
(IVd-1) contacting the reduced catalyst with a second stripping gas to produce the regenerated catalyst.

18. The process of claim 16, wherein step (IVc) is carried out at a temperature of the oxidized precursor catalyst higher than a use temperature of the regenerated catalyst, and step (IVd) further comprises:
(IVd-2) cooling the reduced catalyst or the regenerated catalyst to the use temperature in a duration no greater than 10 minutes.

19. A dehydrogenation process using the regenerated catalyst produced by a process of claim 1, the dehydrogenation process comprising:
(VI) contacting a hydrocarbon-containing feed with the regenerated catalyst to effect one or more of dehydrogenation, dehydroaromatization, and dehydrocyclization of at least a portion of the hydrocarbon-containing feed to produce the at least partially deactivated catalyst comprising the Group 10 element, the inorganic support, and the contaminant and an effluent comprising one or more upgraded hydrocarbons and molecular hydrogen, wherein the hydrocarbon feed comprises one or more of C₂-C₁₆ linear or branched alkanes, one or more of C₄-C₁₆ cyclic alkanes, one or more of C₈-C₁₆ alkyl aromatic hydrocarbons, or a mixture thereof; and
(VII) repeating steps (I) through (IV), wherein, in step (III) additional oxidized precursor catalyst is produced, and wherein, in step (IV), additional regenerated catalyst is obtained from the additional oxidized precursor catalyst; and
(VIII) contacting an additional quantity of the hydrocarbon-containing feed with at least a portion of the additional regenerated catalyst to produce additional at least partially deactivated catalyst and additional effluent.

20. The dehydrogenation process of claim 19, wherein a cycle time from the contacting the hydrocarbon-containing feed with the regenerated catalyst in step (VI) to the contacting the additional quantity of the hydrocarbon-containing feed with the additional regenerated catalyst in step (VIII) is ≤5 hours.

21. A process for upgrading a hydrocarbon, comprising:
(I) contacting a hydrocarbon-containing feed with a catalyst comprising a Group 10 element and an inorganic support to effect one or more of dehydrogenation, dehydroaromatization, and dehydrocyclization of at least a portion of the hydrocarbon-containing feed to produce an at least partially deactivated catalyst comprising the Group 10 element, the inorganic support, and a contaminant and an effluent comprising one or more upgraded hydrocarbons and molecular hydrogen, wherein:
the hydrocarbon-containing feed comprises one or more of C₂-C₁₆ linear or branched alkanes, or one or more of C₄-C₁₆ cyclic alkanes, one or more C₈-C₁₆ alkyl aromatics, or a mixture thereof;
the Group 10 element has a concentration in the range from 0.06 wt % to 6 wt %, based on the weight of the inorganic support;
the hydrocarbon-containing feed and the catalyst are contacted at a temperature in a range from 300° C. to 900° C.; and
the one or more upgraded hydrocarbons comprise at least one of a dehydrogenated hydrocarbon, a dehydroaromatized hydrocarbon, and a dehydrocyclized hydrocarbon;
(II) heating the at least partially deactivated catalyst using a heating gas mixture comprising H₂O at a concentration of greater than 5 mol %, based on the total moles in the heating gas mixture to produce a precursor catalyst;
(III) providing an oxidative gas comprising no greater than 2 mol % of H₂O, based on the total moles in the oxidative gas;
(IV) contacting the precursor catalyst at an oxidizing temperature in a range from 620° C. to 1,000° C. with the oxidative gas for a duration of at least 30 seconds, to produce an oxidized precursor catalyst;
(V) obtaining a regenerated catalyst from the oxidized precursor catalyst; and
(VI) contacting an additional quantity of the hydrocarbon-containing feed with at least a portion of the regenerated catalyst to produce additional at least partially deactivated catalyst and additional effluent.

22. The process of claim 21, wherein the heating gas mixture is produced by combusting a fuel with an oxidizing gas.

23. The process of claim 22, wherein the fuel comprises at least one of H₂, CO, and a hydrocarbon, and the oxidizing gas comprises 02.

24. The process of claim 21, wherein:
the Group 10 element comprises Pt, and
the inorganic support comprises at least 0.5 wt % of a Group 4 element, based on the weight of the inorganic support.

25. The process of claim 21, wherein the catalyst further comprises up to 10 wt % of a promoter, based on the weight of the inorganic support, and wherein the promoter comprises one or more of the following elements: Sn, Ga, Zn, Ge, In, Re, Ag, Au, Cu, a combination thereof, or a mixture thereof.

26. The process of claim 21, wherein the catalyst further comprises up to 5 wt % of an alkali metal element, based on the weight of the inorganic support, and wherein the alkali metal element comprises at least one of: Li, Na, K, Rb, and Cs.

27. The process of claim 21, wherein step (III) comprises:
(IIIa) providing the oxidative gas at a temperature below the oxidizing temperature; and
(IIIb) pre-heating the oxidative gas to a temperature higher than the temperature of the precursor catalyst before the contacting in step (IV).

28. The process of claim 21, further comprising:
(VII) heating the oxidative gas, the precursor catalyst, or both, during step (IV) by using a radiant/conductive heat source, a heat exchanger, or a combination thereof.

29. The process of claim 21, wherein a cycle time from the contacting the hydrocarbon-containing feed with the catalyst in step (I) to the contacting the additional quantity of the hydrocarbon-containing feed with the regenerated catalyst in step (VI) is ≤5 hours.
